SEQUENCE LISTING (3 sequences)

SEQ ID NO: 1 - protein, 6 aa, synthetic construct
IGVAPG

SEQ ID NO: 2 - protein, 6 aa, synthetic construct
VGVAPG

SEQ ID NO: 3 - protein, 6 aa, synthetic construct
VGVTAG

(19) United States
(12) Patent Application Publication
(10) Pub. No.: US 2024/0374618 A1
(43) Pub. Date: Nov. 14, 2024

(54) COMPOSITION OF TANNIC ACID AND VITAMIN D AND METHODS OF USE

(71) Applicant: Nulastin, Inc., Boulder, CO (US)

(72) Inventors: Aleksander HINEK, Toronto (CA); Thomas F. MITTS, Visalia, CA (US)

(21) Appl. No.: 18/510,158

(22) Filed: Nov. 15, 2023

Related U.S. Application Data
(63) Continuation of application No. PCT/US2022/029661, filed on May 17, 2022.
(60) Provisional application No. 63/189,291, filed on May 17, 2021.
(60) Provisional application No. 63/235,889, filed on Aug. 23, 2021.

(51) Int. Cl. A61K 31/593; A61K 9/00; A61K 31/366; A61K 31/7034; A61K 31/728; A61P 19/04

(52) U.S. Cl. CPC A61K 31/593; A61K 9/0014; A61K 31/366; A61K 31/7034; A61K 31/728; A61P 19/04

(57) ABSTRACT

Compositions, kits, and methods that can be used to improve elastin concentration are described herein. For example, pharmaceutical compositions described herein comprise a therapeutically effective amount of a first agent and a therapeutically effective amount of a second agent, wherein the first agent is tannic acid, ellagic acid, or combinations thereof, and the second agent is vitamin D, hyaluronic acid, ferulic acid, or combinations thereof.

### CROSS-REFERENCE

This application is a continuation of International Patent Application No. PCT/US2022/029661, filed May 17, 2022, which claims the benefit of U.S. Provisional Patent Application No. 63/235,889, filed Aug. 23, 2021, and U.S. Provisional Patent Application No. 63/189,291, filed on May 17, 2021, which is incorporated herein by reference in its entirety.

### BACKGROUND

Elastin is a crucial component of the structural scaffolding of connective tissues, allowing connective tissue to stretch under mechanical stress and return to an unstretched state without damage. Elastin is synthesized by fibroblasts, myoblasts, and other cell types, in the form of precursor monomers called tropoelastin. Tropoelastin is secreted into the extracellular matrix, aligned along preexisting microfibrillar scaffolds, and eventually enzymatically polymerized into insoluble elastin, a stable component of the extracellular elastic fibers that are mechanistically responsible for tissue adaptation.

During the normal aging process, and in other pathologic situations, the stretchable elastic fibers can also be proteolytically degraded and replaced by stiff collagen polymers. Reconstruction of the damaged tissues involves large-scale production of new extracellular matrix components, resulting in the formation of scar tissues in which elastic fibers are scarce or absent. The inability of new elastic fiber synthesis can lead to sagging skin, degenerative progression of cardiac fibrosis, or development of high blood pressure.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1A depicts the overlay of the fluorescence channel that highlights elastin fibers and the fluorescence channel that highlights fibroblast cells.

FIG. 1B depicts only the fluorescence channel that highlights the elastin fibers.

FIG. 1C depicts only the fluorescence channel that highlights the fibroblast cells.

FIG. 2A depicts the amount of tropoelastin in fibroblast cultures measured using PCR after 18 hours. Adding tannic acid to cultures that contained vitamin D3 increased the amount of tropoelastin present in the fibroblast cultures

FIG. 2B depicts the amount of insoluble elastin in fibroblast cultures measured after 72 hours.

FIG. 3A depicts the overlay of the fluorescence channel that highlights elastin fibers and the fluorescence channel that highlights fibroblast cells.

FIG. 3B depicts only the fluorescence channel that highlights the elastin fibers.

FIG. 3C depicts only the fluorescence channel that highlights the fibroblast cells.

FIG. 4A depicts the overlay of the fluorescence channel that highlights the collagen matrix and the fluorescence channel that highlights fibroblast cells.

FIG. 4B depicts only the fluorescence channel that highlights the collagen matrix.

FIG. 4C depicts only the fluorescence channel that highlights the fibroblast cells.

FIG. 5A depicts the overlay of the fluorescence channel that highlights elastin fibers and the fluorescence channel that highlights fibroblast cells.

FIG. 5B depicts only the fluorescence channel that highlights the elastin fibers.

FIG. 5C depicts only the fluorescence channel that highlights the fibroblast cells.

FIG. 6A displays morphometry of elastin.

FIG. 6B depicts a result of an assay of the amount of insoluble elastin present in the cultures.

FIG. 7A depicts the overlay of the fluorescence channel that highlights the collagen matrix and the fluorescence channel that highlights fibroblast cells.

FIG. 7B depicts only the fluorescence channel that highlights the collagen matrix.

FIG. 7C depicts only the fluorescence channel that highlights the fibroblast cells.

FIG. 8 is a graph of the quantitation of collagen I in human derived fibroblast cultures treated with 5 μg/mL ferulic acid, 2 μg/mL tannic acid, or both 5 μg/mL ferulic acid and 2 μg/mL tannic acid.

FIG. 9A depicts the overlay of the fluorescence channel that highlights elastin fibers and the fluorescence channel that highlights fibroblast cells.

FIG. 9B depicts only the fluorescence channel that highlights the elastin fibers.

FIG. 9C depicts only the fluorescence channel that highlights the fibroblast cells.

### SUMMARY

In some embodiments disclosed herein are compositions comprising a) a first agent that blocks elastin production; and b) a second agent that promotes elastin production, wherein the second agent is present at a concentration of 0.1 nM to 100 nM.

In some embodiments, the second agent neutralizes blocking of elastin production by the first agent. In some embodiments, the first agent is a moisturizing agent. In some embodiments, the moisturizing agent is hyaluronic acid. In some embodiments, the hyaluronic acid is present in the composition at a concentration of about 10 μg/mL to about 100 μg/mL. In some embodiments, the moisturizing agent is ferulic acid. In some embodiments, the ferulic acid is present in the composition at a concentration of about 1 μg/mL to about 15 μg/mL. In some embodiments, the moisturizing agent is vitamin D. In some embodiments, the vitamin D is vitamin D3. In some embodiments, the vitamin D is present in the composition at a concentration of about 0.01 wt % to about 10 wt %.

In some embodiments, the second agent is a polyphenol. In some embodiments, the polyphenol is tannic acid. In some embodiments, the polyphenol is ellagic acid. In some embodiments, the second agent is present in the composition at a concentration of at most 50 nM. In some embodiments, the second agent is present in the composition at a concentration of at most 10 nM. In some embodiments, the second agent is present in the composition at a concentration of at most 2 nM. In some embodiments, the second agent is present in the composition at a concentration of about 1 μg/mL to about 15 μg/mL.

In some embodiments, the composition further comprises an excipient. In some embodiments, the composition further comprises a filler. In some embodiments, the composition further comprises a disintegrant. In some embodiments, the composition further comprises a preservative agent. In some embodiments, the composition further comprises an antioxidant. In some embodiments, the composition further comprises a solvent. In some embodiments, the composition further comprises a preservative. In some embodiments, the composition further comprises a suspending agent. In some embodiments, the composition further comprises a surfactant. In some embodiments, the composition further comprises a binder. In some embodiments, the composition further comprises a diluent. In some embodiments, the composition further comprises a lubricant. In some embodiments, the composition further comprises a wetting agent. In some embodiments, the composition further comprises a glidant. In some embodiments, the composition further comprises a stabilizing agent. In some embodiments, the composition further comprises a dispersing agent.

In some embodiments, the composition further comprises an elastin-derived peptide. In some embodiments, the elastin-derived peptide is selected from the group consisting of IGVAPG (SEQ ID NO: 1),In some embodiments, the elastin-derived peptide is selected from the group consisting of VGVAPG (SEQ ID NO: 2). In some embodiments, the elastin-derived peptide is selected from the group consisting of VGVTAG (SEQ ID NO: 3).

In some embodiments, the composition further comprises water. In some embodiments, the composition is substantially free of water.

In some embodiments, the composition is formulated as a cream. In some embodiments, the composition is formulated as an injectable.

In some embodiments disclosed herein are methods of enhancing elastin formation comprising: administering to a subject in need thereof a therapeutically-effective amount of a composition, wherein the composition comprises: i) a first agent that blocks elastin production; and ii) a second agent that promotes elastin production, wherein the second agent is present at a concentration of 0.1 nM to 100 nM.

In some embodiments, the second agent neutralizes blocking of elastin production by the first agent. In some embodiments, the first agent is a moisturizing agent. In some embodiments, the moisturizing agent comprises hyaluronic acid. In some embodiments, the hyaluronic acid is present in the composition at a concentration of about 10 μg/mL to about 100 μg/mL. In some embodiments, the moisturizing agent comprises ferulic acid. In some embodiments, the ferulic acid is present in the composition at a concentration of about 1 μg/mL to about 15 μg/mL. In some embodiments, the moisturizing agent comprises vitamin D. In some embodiments, the vitamin D is vitamin D₃. In some embodiments, the vitamin D is present in the composition at a concentration of about 0.01 wt % to about 10 wt %.

In some embodiments, second agent is a polyphenol. In some embodiments, the polyphenol is tannic acid. In some embodiments, the polyphenol is ellagic acid. In some embodiments, the second agent is present in the composition at a concentration of at most 50 nM. In some embodiments, the second agent is present in the composition at a concentration of at most 10 nM. In some embodiments, the second agent is present in the composition at a concentration of at most 2 nM. In some embodiments, the second agent is present in the composition at a concentration of about 1 μg/mL to about 15 μg/mL.

In some embodiments, the composition administered to the subject further comprises an excipient. In some embodiments, the composition administered to the subject further comprises a filler. In some embodiments, the composition administered to the subject further comprises a disintegrant. In some embodiments, the composition administered to the subject further comprises a preservative agent. In some embodiments, the composition administered to the subject further comprises an antioxidant. In some embodiments, the composition administered to the subject further comprises a solvent. In some embodiments, the composition administered to the subject further comprises a preservative. In some embodiments, the composition administered to the subject further comprises a suspending agent. In some embodiments, the composition administered to the subject further comprises a surfactant. In some embodiments, the composition administered to the subject further comprises a binder. In some embodiments, the composition administered to the subject further comprises a diluent. In some embodiments, the composition administered to the subject further comprises a lubricant. In some embodiments, the composition administered to the subject further comprises a wetting agent. In some embodiments, the composition administered to the subject further comprises a glidant. In some embodiments, the composition administered to the subject further comprises a stabilizing agent. In some embodiments, the composition administered to the subject further comprises a dispersing agent.

In some embodiments, the composition administered to the subject further comprises an elastin-derived peptide. In some embodiments, the elastin-derived peptide is selected from the group consisting of IGVAPG (SEQ ID NO: 1). In some embodiments, the elastin-derived peptide is selected from the group consisting of VGVAPG (SEQ ID NO: 2). In some embodiments, the elastin-derived peptide is selected from the group consisting of VGVTAG (SEQ ID NO: 3).

In some embodiments, the composition administered to the subject further comprises water. In some embodiments, the composition administered to the subject is substantially free of water.

In some embodiments, the administering is topical. In some embodiments, the administering is by injection.

In some embodiments, the administering enhances elastin formation in the subject by at least 25% as compared to administration of a control, wherein the control is an analogous composition that lacks the second agent. In some embodiments, the administering enhances collagen formation by at least 25% as compared to the control. In some embodiments, the subject is a mammal. In some embodiments, the subject is a human.

In some embodiments disclosed herein are methods of enhancing elastin formation, the method comprising: administering to a subject in need thereof a therapeutically-effective amount of a composition, wherein the composition comprises: i) a first agent that blocks elastin production; and ii) a second agent that promotes elastin production, wherein the administering enhances elastin formation by at least 25% as compared to administration of a control, wherein the control is an analogous composition that lacks the second agent.

In some embodiments disclosed herein are methods of enhancing elastin formation, the method comprising: administering to a subject in need thereof a therapeutically-effective amount of a composition, wherein the composition comprises: i) a first agent that blocks elastin production; and a second agent that promotes elastin production, wherein the administering enhances collagen formation by at least 25% as compared to administration of a control, wherein the control is an analogous composition that lacks the second agent.

In some embodiments disclosed herein are compositions comprising: a) vitamin D and b) tannic acid, wherein the tannic acid is present at a concentration from 0.1 nM to 100 nM.

### DETAILED DESCRIPTION

Various compositions, methods, and kits are described herein for the treatment of a subject in need of improved elastin concentration. The methods can include administration of at least one pharmaceutical composition to the subject. The compositions and methods can maintain or improve elastin concentration during treatment with compounds associated with a reduction of elastin concentration.

### Compositions

Treatment of tissue damage in skin and vascular tissue involves using various pharmaceutical compositions to improve tissue health while decreasing the damaging effects of age, cardiovascular disease, and environmental irritants. Damaged skin and vascular tissue can cause health problems if not corrected.

Skin is the outer layer of tissue covering the body of a person or animal. Skin, also known as dermal tissue, is made of multiple ectodermal tissue layers such as the epidermis, the dermis, and the hypodermis. The dermis, the middle layer of the skin, is comprised of a fibrous network of collagen, elastic tissue, and other extracellular components including vasculature, nerve endings, hair follicles, and glands. The dermis protects the skin's deeper layers and assists in both sensation and thermoregulation.

The vascular system, also called the circulatory system, is made up of the vessels, such as arteries and veins, that carry blood and lymph through the body. The extracellular matrix surrounding vascular cells consists of elastin, collagen, and other proteins that provide mechanical stability and are crucial for vascular function and homeostasis.

Connective tissue, dermal tissue, vascular tissue, lungs, and intestines all have high concentrations of elastin and collagen, which maintain elasticity and flexibility. Elastin and collagen are also important components in cardiac and pulmonary tissue and allow that blood vessels and lungs recover from repeated mechanical stress. Loss of elastin and collagen are associated with health complications such as emphysema, atherosclerosis, hypertension, and others.

Common formulations used to treat tissue damage such as sun damage, scar tissue, or age-related problems, also inhibit elastin formation and deposition. Non-limiting examples of formulations that treat tissue damage but can also inhibit elastin formation include vitamin D, hyaluronic acid, and ferulic acid. Vitamin D can protect skin against ultraviolet light damage. Hyaluronic acid is a hydrating agent that can increase the moisture content of stressed or ageing tissue. Ferulic acid is a powerful antioxidant and free radical scavenger and can protect against tissue damage. These compounds are beneficial but can also reduce elastin content in tissue. Both vitamin D, along with tannic acid, can increase the production of fibronectin and fibrillin. However, ferulic acid can block the production of fibronectin and fibrillin and can decrease fibroblast mobility and elastin production. To overcome the inhibition of elastin formation of formulations such as vitamin D, hyaluronic acid, ferulic acid, a polyphenolic compound can be added to provide benefits to the skin, cardiac tissue, and arterial tissue in improving elastin production.

Elastin is an insoluble, fibrous protein that, in humans, is encoded by the ELN gene. Elastin is a key component of the extracellular matrix, where elastin forms randomly oriented interconnected fibrous systems. Elastin fibers have structures with shape memory that are mainly responsible for the ability of skin and other tissues to return to a prior shape when distorted by stretching or contracting. Elastin is also an important load bearing-tissue and can store mechanical energy.

Elastin is responsible for skin's essential elasticity and tonicity. Elastin's thin, rubbery structure provides these properties. Biosynthesis of elastin begins with the embryonic period and continues through adulthood, at which time the body stops producing elastin. Thus, elastin is no longer renewed. With aging, the elastic fibers progressively degenerate and separate into fragments. The skin progressively loses elasticity and forms fine lines and wrinkles. The skin aging process can be accelerated by environmental damage such as radiation or pollution.

Elastic fibers are composed of elastin and microfibrils, which are composed of a mixture of elastin and glycoproteins, including fibrillin. Elastin fiber formation (elastogenesis) is a complex process involving intracellular and extracellular events. Cells such as fibroblasts, endothelial cells, chondroblasts, mycardiocytes, or vascular smooth muscle cells, first synthesize and secrete glycoproteins that form a microfibrillar scaffold into the extracellular space. Tropoelastin, the soluble precursor peptide of elastin, is synthesized in these cells by ribosomes in the rough endoplasmic reticulum and transported throughout the Golgi apparatus and secretory vesicles that deposit tropoelastin in the extracellular space.

Once in the extracellular space, Soluble tropoelastin monomers are processed into elastin through the cleaving of a signal peptide and self-aggregation into elastin fibers. Hydrophobic domains of tropoelastin are responsible for self-assembly into elastin. Cross-linking domains of tropoelastin are defined by desmosine, an amino acid formed from condensation of lysine fragments. These domains are responsible for the formation of durable cross-links to form the coils of tropoelastin. The coils form a spring-like secondary structure that gives tropoelastin the ability to stretch. Lysyl oxidase aids in the cross-linking of tropoelastin molecules and leads to elastin formation.

Collagen is the main structural protein of the extracellular matrix in connective tissues and makes up about one-third of the protein in the human body. Collagen is typically made by fibroblast cells. Collagens form fibrous matrices in connective tissue such as cartilage, bones, tendons, ligaments, and skin. Collagen provides most of the tensile strength of tissue.

Elastin and collagen fibers form mesh-like networks in the extracellular space. Some compounds can inhibit the formation or deposition of elastin or collagen. Non-limiting examples of elastin-or collagen-blocking agents include vitamin D, hyaluronic acid, and ferulic acid. These agents can inhibit elastin production during treatment to provide an undesired decrease in elasticity of the tissue. Vitamin D, hyaluronic acid, and ferulic acid can also all act as moisturizing agents or emollients, compounds that protect, moisturize, and lubricate skin.

Vitamin D is a group of fat-soluble secosteroids responsible for increasing absorption of calcium, magnesium, and phosphate. Vitamin D possesses anti-inflammatory, anti-oxidant, and neuroprotective properties. Vitamin D includes vitamin D₁, vitamin D₂, vitamin D₃, vitamin D₄, vitamin D₅-, and analogues of such. Vitamin D, which improves cardiovascular function and protects against sun damage, can be used both systemically and topically.

Vitamin D compounds include, but are not limited to, vitamins D₂ (ergocalciferol) and D₃ (cholecalciferol) (sometimes collectively referred to as “calciferol”); compounds and isomers and derivatives of these compounds, such as vitamin D₂ and D₃ prohormones (e.g., 25-hydroxyvitamin D₂ (250HD2) and 25-hydroxyvitamin D₃ (250HD3), collectively “calcidiol”); active vitamin D₂ and D₃ (e.g., 1,25-dihydroxyvitamin D₂ (1,25(OH)2) and 1,25-dihydroxyvitamin D₃ (1,25(OH)2D3), collectively “calcitriol”); metabolites of these compounds; and combinations of the above. Vitamin D compounds also include less common vitamin D compounds such as vitamin D₁ compounds, vitamin D₂ compounds, vitamin D₄ compounds, and synthetic versions of the above-mentioned vitamin D compounds.

Hyaluronic acid (HA) is a non-sulfated glycosaminoglycan that is distributed throughout connective, epithelial, and neural tissues. HA is a main component of the extracellular matrix and is important to cell proliferation and migration. Hyaluronic acid is a potent moisturizer and can be used in topical treatments or injections to decrease wrinkles and improve dermal health.

A sample of hyaluronic acid can include an anionic, non-sulfated glycosaminoglycan polymer with disaccharide units, which include D-glucuronic acid and D-N-acetylglucosamine monomers, linked together via alternating β-1,4 and β-1,3 glycosidic bonds or a pharmaceutically acceptable salt thereof. Hyaluronic acid polymers can be purified from animal and non-animal sources. Polymers of hyaluronic acid can range in size from about 5,000 Da to about 20,000,000 Da. Any hyaluronic acid polymer is useful in the compositions disclosed herein. In some embodiments, the hyaluronic acid improves a condition of the skin. Non-limiting examples of pharmaceutically acceptable salts of hyaluronic acid include sodium hyaluronate, potassium hyaluronate, magnesium hyaluronate, calcium hyaluronate, and combinations thereof.

Ferulic acid is a hydroxycinnamic acid common in plants. Ferulic acid is an antioxidant and can exhibit anti-inflammatory properties.

Although some compounds can inhibit or block the formation of extracellular matrix proteins such as elastin or collagen, other compounds can work counter to this inhibition by promoting elastin and collagen production or by neutralizing or reactivating the blocked elastin production. Non-limiting examples of compounds that promote elastin and collagen production are polyphenols, such as tannic acid and ellagic acid.

Polyphenols are organic compounds that act as micronutrients and naturally occur in plants. Polyphenols can be antioxidants, which prevent or reverse damage in cells caused by aging, the environment, and lifestyle attributes. Polyphenols are generally compounds bearing several hydroxyl groups on aromatic rings.

Ellagic acid and tannic acid are polyphenols found in a wide variety of fruits and nuts such as raspberries, strawberries, walnuts, grapes, and black currants. These molecules possess potent ability to scavenge reactive oxygen species (ROS) and reactive nitrogen species (RNS). Both ROS and RNS, generated inside cells after exposure to several endogenous and exogenous agents, cause direct or indirect damage to many important biomolecules, including elastin mRNA, by activation of local proteinases, glycosidases, or RNAses.

Tannic acid is a specific form of tannin, a class of astringent polyphenols that bind to and precipitate other proteins. Tannins are widely found in plants and protect against predation and assist in regulating plant growth. Tannic acid is weakly acidic with a pKₐ of about 6. Tannic acid exists in two forms—quercitannic acid and gallotannic acid. Tannic acid possesses both antimicrobial and antioxidant properties.

Ellagic acid is a dilactone of hexahydroxydiphenic acid. Ellagic acid is produced by plants through the hydrolysis of tannins such as ellagitannin and geraniin.

A polyphenol, for example, tannic acid or ellagic acid, in combination with moisturizing agents such as vitamin D, hyaluronic acid, or ferulic acid can neutralize or reverse the undesired effects of the moisturizing agents on the production of elastin, collagen, or both. In the presence of tannic acid or ellagic acid, treatments that would otherwise lead to reduction in elastin and collagen can be used without affecting these connective tissue components.

### Pharmaceutical Compositions

Compositions can comprise a first agent that blocks elastin production and a second agent that promotes elastin production. Compositions can be therapeutically effective, cosmetically effective, or both. In some embodiments, a therapeutically-effective amount of a composition is a cosmetically-effective amounts. In some embodiments, a therapeutically-effective amount of a composition and a cosmetically-effective amount of a composition are different amounts.

In some embodiments, the first agent is a polyphenol or polyphenolic acid. Non-limiting examples of polyphenolic acids include tannic acid, ellagic acid, theaflavin, luteolin, quercetin, kaempferol, resveratrol, apigenin, catechin, apigenin, curcumin, berberine, naringenin, myricetin, rutin, hesperidin, and combinations thereof. In some embodiments, the first agent is tannic acid, ellagic acid, or a combination thereof. In some embodiments, the first agent is tannic acid. In some embodiments, the first agent is ellagic acid. In some embodiments, the first agent is a combination of tannic acid and ellagic acid.

In some embodiments, the second agent is a moisturizing agent such as vitamin D, hyaluronic acid, ferulic acid, or a combination thereof. In some embodiments, the second agent is vitamin D. In some embodiments, the second agent is vitamin D₃. In some embodiments, the second agent is hyaluronic acid. In some embodiments, the second agent is ferulic acid. In some embodiments, the second agent is vitamin D and hyaluronic acid, vitamin D and ferulic acid, hyaluronic acid and ferulic acid, or a combination of all three: vitamin D, hyaluronic acid, and ferulic acid.

For illustration, Table 1 shows various combinations of the first agent and the second agent.

TABLE 1

| No. | First agent | Second agent |
|---|---|---|
| 1 | Tannic acid | Vitamin D |
| 2 | Tannic acid | Hyaluronic acid |
| 3 | Tannic acid | Ferulic acid |
| 4 | Ellagic acid | Vitamin D |
| 5 | Ellagic acid | Hyaluronic acid |
| 6 | Ellagic acid | Ferulic acid |
| 7 | Tannic acid and Ellagic acid | Vitamin D |
| 8 | Tannic acid and Ellagic acid | Hyaluronic acid |
| 9 | Tannic acid and Ellagic acid | Ferulic acid |
| 10 | Tannic acid | Vitamin D and Hyaluronic acid |
| 11 | Ellagic acid | Vitamin D and Hyaluronic acid |
| 12 | Tannic acid and Ellagic acid | Vitamin D and Hyaluronic acid |
| 13 | Tannic acid | Vitamin D and Ferulic acid |
| 14 | Ellagic acid | Vitamin D and Ferulic acid |
| 15 | Tannic acid and Ellagic acid | Vitamin D and Ferulic acid |
| 16 | Tannic acid | Hyaluronic acid and Ferulic acid |
| 17 | Ellagic acid | Hyaluronic acid and Ferulic acid |
| 18 | Tannic acid and Ellagic acid | Hyaluronic acid and Ferulic acid |
| 19 | Tannic acid | Vitamin D and Hyaluronic acid |
| | | and Ferulic acid |
| 20 | Ellagic acid | Vitamin D and Hyaluronic acid |
| | | and Ferulic acid |
| 21 | Tannic acid and Ellagic acid | Vitamin D and Hyaluronic acid |
| | | and Ferulic acid |

In some embodiments, the first agent is tannic acid, and the second agent is vitamin D. In some embodiments, the first agent is tannic acid, and the second agent is hyaluronic acid. In some embodiments, the first agent is tannic acid, and the second agent is ferulic acid.

In some examples, the pharmaceutical composition comprises a tannic acid present in an amount of about 1 μg/mL to about 15 μg/mL, about 1 μg/mL to about 10 μg/mL, about 1 μg/mL to about 5 μg/mL, or about 1 μg/mL to about 2 μg/mL. Examples can include about 1 μg/mL, about 2 μg/mL, about 3 μg/mL, about 4 μg/mL, about 5 μg/mL, about 6 μg/mL, about 7 μg/mL, 8 μg/mL, 9 μg/mL, 10 μg/mL, 11 μg/mL, 12 μg/mL, 13 μg/mL, 14 μg/mL, and 15 μg/mL. In some examples, the first agent is tannic acid present in an amount of about 2 μg/mL.

In some embodiments, the polyphenol of the pharmaceutical composition can be present at a nM concentration of at least 0.01, at least 0.1, at least 0.2, at least 0.3, at least 0.4, at least 0.5, at least 0.6, at least 0.7, at least 0.8, at least 0.9, at least 1, at least 2, at least 3, at least 4, at least 5, at least 6, at least 7, at least 8, at least 9, at least 10, at least 15, at least 20, at least 30, at least 40, at least 50, at least 60, at least 70, at least 80, at least 90, at least 100, at least 150, at least 200, at least 300, at least 400, or at least 500 nM. In some embodiments, the polyphenolic compound of the pharmaceutical composition to promote elastin production can be present at a concentration of at most 500, at most 400, at most 300, at most 200, at most 150, at most 100, at most 90, at most 80, at most 70, at most 60, at most 50, at most 40, at most 30, at most 20, at most 15, at most 10, at most 9, at most 8, at most 7, at most 6, at most 5, at most 4, at most 3, at most 2, at most 1, at most 0.9, at most 0.8, at most 0.7, at most 0.6, at most 0.5, at most 0.4, at most 0.3, at most 0.2, at most 0.1, or at most 0.01 nM.

In some embodiments, the pharmaceutical composition affects elastin or collagen production of cells. Cells can be epithelial cells, fibroblasts, smooth muscle cells, chondrocytes, or endothelial cells. In some embodiments, cells are fibroblast cells. Fibroblasts are cells that synthesize the extracellular matrix and collagen. Fibroblast cells produce the structural framework for animal tissues and play a critical role in wound healing.

In some embodiments, the pharmaceutical composition comprises a first agent, wherein the first agent is tannic acid. In some embodiments, the highest concentration of tannic acid in the skin after administration of one dose of the pharmaceutical composition (the Cₘₐₓ) is about 0.1 micromolar to about 5 micromolar.

In some embodiments, the pharmaceutical composition comprises a second agent, wherein the second agent is hyaluronic acid present in an amount of about 10 μg/mL to about 100 μg/mL, about 20 μg/mL to about 90 μg/mL, about 30 μg/mL to about 80 μg/mL, about 40 μg/mL to about 70 μg/mL, or about 50 μg/mL to about 60 μg/mL. Examples include about 10 μg/mL, about 20 μg/mL, about 30 μg/mL, about 40 μg/mL, about 50 μg/mL, about 60 μg/mL, about 70 μg/mL, about 80 μg/mL, about 90 μg/mL, and about 100 μg/mL. In some embodiments, the pharmaceutical composition comprises hyaluronic acid present in an amount of about 0.01 wt. % to about 85 wt. % of the composition, about 0.01 wt. % to about 70 wt. % of the composition, about 0.01 wt. % to about 60 wt. % of the composition, about 0.01 wt. % to about 40 wt. % of the composition, about 0.01 wt. % to about 20 wt. % of the composition, or about 0.01 wt. % to about 10 wt. %. In some embodiments, the pharmaceutical composition comprises hyaluronic acid in an amount of about 0.1 wt. %. In some embodiments, the Cₘₐₓ of hyaluronic acid in the pharmaceutical composition is about 1 micromolar to about 50 micromolar.

In some embodiments, the pharmaceutical composition comprises ellagic acid in an amount of about 1 μg/mL to about 15 μg/mL, about 1 μg/mL to about 10 μg/mL, about 1 μg/mL to about 5 μg/mL, or about 1 μg/mL to about 2 μg/mL. Examples include about 1 μg/mL, about 2 μg/mL, about 3 μg/mL, about 4 μg/mL, about 5 μg/mL, about 6 μg/mL, about 7 μg/mL, about 8 μg/mL, about 9 μg/mL, about 10 μg/mL, about 11 μg/mL, about 12 μg/mL, about 13 μg/mL, about 14 μg/mL, and about 15 μg/mL. In some embodiments, first compound is ellagic acid present in an amount of about 2 μg/mL. In some embodiments, the Cₘₐₓ of ellagic acid in the pharmaceutical composition is about 0.1 micromolar to about 5 micromolar.

In some embodiments, the pharmaceutical composition comprises a second agent, wherein the second agent is vitamin D present in an amount of about 0.01 wt. % to about 10 wt. % of the composition, about 0.01 wt. % to about 8 wt. % of the composition, about 0.01 wt. % to about 6 wt. % of the composition, about 0.01 wt. % to about 4 wt. % of the composition, about 0.01 wt. % to about 2 wt. % of the composition, or about 0.01 wt. % to about 1 wt. % of the composition. Examples include about 0.01 wt. %, about 0.1 wt. %, about 1 wt. %, about 2 wt. %, about 5 wt. %, about 9 wt. %, and about 10 wt. %. In some embodiments, the pharmaceutical composition comprises a therapeutically effective amount of a second agent, wherein the second agent is vitamin D present in an amount about 10 μg/mL to about 100 μg/mL, about 20 μg/mL to about 90 μg/mL, about 30 μg/mL to about 80 μg/mL, about 40 μg/mL to about 70 μg/mL, or about 50 μg/mL to about 60 μg/mL. Examples include about 10 μg/mL, about 20 μg/mL, about 30 μg/mL, about 40 μg/mL, about 50 μg/mL, about 60 μg/mL, about 70 μg/mL, about 80 μg/mL, about 90 μg/mL, and about 100 μg/mL. In some embodiments, the Cₘₐₓ of vitamin D in the pharmaceutical composition is about 1 micromolar to about 50 micromolar.

In some embodiments, the pharmaceutical composition comprises a therapeutically effective amount of a second agent, wherein the second agent is ferulic acid present in an amount about 1 μg/mL to about 15 μg/mL, about 1 μg/mL to about 10 μg/mL, about 1 μg/mL to about 5 μg/mL, or about 1 μg/mL to about 2 μg/mL. Examples include about 1 μg/mL, about 2 μg/mL, about 3 μg/mL, about 4 μg/mL, about 5 μg/mL, about 6 μg/mL, about 7 μg/mL, about 8 μg/mL, about 9 μg/mL, about 10 μg/mL, about 11 μg/mL, about 12 μg/mL, about 13 μg/mL, about 14 μg/mL, and about 15 μg/mL. In some embodiments, the pharmaceutical composition comprises a therapeutically-effective amount of a first compound and a therapeutically-effective amount of a second compound, wherein the second compound is ferulic acid present in an amount of about 20 μM or about 3.88 μg/mL. In some embodiments, the Cₘₐₓ of ferulic acid in the pharmaceutical composition is about 0.1 micromolar to about 55 micromolar.

In some embodiments, the pharmaceutical composition further comprises elastin, elastogenic peptides, or elastin-derived peptides. Elastin-derived peptides can stimulate the production and assembly of tropoelastin into elastin fibers. In some embodiments, the elastogenic peptides are modified peptides such that any amino acid residue of the elastogenic peptide carries a substitution selected from acetyl, amide, carbonyl, imide, thiol, alkyl, alkenyl, alkynyl, cycloalkyl, sulfonyl, sulfinyl, silyl, a fatty acid, pyroglutamyl, isocyanate, alkyl carbonyl, alkyl, cycloalkyl, thioester, urea, carbamate, sulfonamide, alkylamine, aryl, alkylaryl, heteroaryl, alkyheteroaryl, furazanyl, furyl, imidazolidinyl, imidazolyl, imidazolinyl, isothiazolyl, isoxazolyl, morpholinyl, oxazolyl, and piperazinyl. In some embodiments, the elastogenic peptides are substituted with D-amino acids. In some embodiments, the elastogenic peptides are acetylated. In some embodiments, the elastogenic peptides are acetylated at the N-terminus. Elastogenic peptides can be elastin protein fragments (e.g., IGVAPG (SEQ ID NO: 1), VGVAPG (SEQ ID NO: 2), or VGVTAG (SEQ ID NO: 3)). In some embodiments, elastogenic peptides are present in the pharmaceutical composition in an amount of about 10 μg/ml, about 20 μg/ml, about 30 μg/ml, about 40 μg/ml, about 50 μg/ml, about 60 μg/ml, about 70 μg/ml, about 80 μg/ml, about 90 μg/ml, or about 100 μg/ml. Alternatively or in addition to the addition of elastogenic peptides, peptides of insulin-like growth factor 1 (IGF-1) or glucagon-like peptide 1 (GLP-1) can be used in the pharmaceutical composition.

In some embodiments, the pharmaceutical composition further comprises an antioxidant, a copper compound, a divalent manganese compound, a trivalent iron compound, aldosterone, metformin, or sodium ascorbate.

Non-limiting examples of copper compounds include copper sulfate, copper phosphate, copper chloride, copper iodide, and copper oxide. A copper compound can be present in a pharmaceutical composition at an amount from about 2 wt. % to about 20 wt. %, from about 2 wt. % to about 10 wt. %, from about 2 wt. % to about 8 wt. %, from about 2 wt. % to about 6 wt. %, or from about 2 wt. % to about 4 wt. % of the composition. A copper compound can be present at about 2 wt. %, about 3 wt. %, about 4 wt. %, about 5 wt. %, about 6 wt. %, about 7 wt. %, about 8 wt. %, about 9 wt. %, about 10 wt. %, about 12 wt. %, about 14 wt. %, about 16 wt. %, about 18 wt. %, or about 20 wt. %.

Non-limiting examples of divalent manganese compounds include manganese ascorbate, manganese-PCA (a manganese salt of L-pyrrolidone carboxylic acid), manganese chloride, manganese nitrate, manganese sulfate, and manganese gluconate. A divalent manganese compound can be present from about 2 wt. % to about 20 wt. %, from about 2 wt. % to about 10 wt. %, from about 2 wt. % to about 8 wt. %, from about 2 wt. % to about 6 wt. %, or from about 2 wt. % to about 4 wt. % of the composition. A divalent manganese compound can be present in about 2 wt. %, about 3 wt. %, about 4 wt. %, about 5 wt. %, about 6 wt. %, about 7 wt. %, about 8 wt. %, about 9 wt. %, about 10 wt. %, about 12 wt. %, about 14 wt. %, about 16 wt. %, about 18 wt. %, or about 20 wt. %. In some embodiments, the divalent manganese compound is present at a concentration of about 0.5 mM, about 1 mM, about 5 mM, about 10 mM, about 15 mM, about 20 mM, about 30 mM, about 40 mM, about 50 mM, about 60 mM, about 70 mM, about 80 mM, about 90 mM, or about 100 mM.

Non-limiting examples of trivalent iron compounds include ferric ammonium citrate and ferric chloride. A trivalent iron compound can be present in a pharmaceutical composition at an amount from about 2 wt. % to about 20 wt. %, from about 2 wt. % to about 10 wt. %, from about 2 wt. % to about 8 wt. %, from about 2 wt. % to about 6 wt. %, or from about 2 wt. % to about 4 wt. % of the composition. A trivalent iron compound can be present at about 2 wt. %, about 3 wt. %, about 4 wt. %, about 5 wt. %, about 6 wt. %, about 7 wt. %, about 8 wt. %, about 9 wt. %, about 10 wt. %, about 12 wt. %, about 14 wt. %, about 16 wt. %, about 18 wt. %, or about 20 wt. %. In some embodiments, the trivalent iron compound is present at a concentration of about 0.5 mM, about 1 mM, about 5 mM, about 10 mM, about 15 mM, about 20 mM, about 30 mM, about 40 mM, about 50 mM, about 60 mM, about 70 mM, about 80 mM, about 90 mM, or about 100 mM.

In some embodiments, aldosterone is present in the pharmaceutical composition at an amount from about 2 wt. % to about 20 wt. %, from about 2 wt. % to about 10 wt. %, from about 2 wt. % to about 8 wt. %, from about 2 wt. % to about 6 wt. %, or from about 2 wt. % to about 4 wt. % of the composition. Aldosterone can be present in about 2 wt. %, about 3 wt. %, about 4 wt. %, about 5 wt. %, about 6 wt. %, about 7 wt. %, about 8 wt. %, about 9 wt. %, about 10 wt. %, about 12 wt. %, about 14 wt. %, about 16 wt. %, about 18 wt. %, or about 20 wt. %. In some embodiments, aldosterone is present at a concentration of about 1 uM, about 2 uM, about 3 uM, about 4 uM, about 5 uM, about 6 uM, about 7 uM, about 8 uM, about 9 uM, about 10 uM, about 20 uM, about 30 uM, about 40 uM, about 50 uM, about 60 uM, about 70 uM, about 80 uM, about 90 uM, about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 2 mM, about 3 mM, about 4 mM, about 5 mM, about 6 mM, about 7 mM, about 8 mM, about 9 mM, or about 10 mM.

Non limiting examples of metformin can include free metformin or a metformin salt such as metformin hydrochloride, metformin phosphate, metformin sulfate, metformin hydrobromide, metformin salicylate, metformin maleate, metformin benzoate, metformin succinate, metformin ethanesulfonate, metformin fumarate, glycolate salts of metformin, metformin orotate, metformin clofibrate, metformin acetylsalicylate, metformin nicotinate, metformin adamantoate, and a sulfamindo aryloxyalkyl carboxylic acid salt of metformin. In some embodiments, metformin is present from about 2 wt. % to about 20 wt. %, from about 2 wt. % to about 10 wt. %, from about 2 wt. % to about 8 wt. %, from about 2 wt. % to about 6 wt. %, or from about 2 wt. % to about 4 wt. % of the composition. Metformin can be present at about 2 wt. %, about 3 wt. %, about 4 wt. %, about 5 wt. %, about 6 wt. %, about 7 wt. %, about 8 wt. %, about 9 wt. %, about 10 wt. %, about 12 wt. %, about 14 wt. %, about 16 wt. %, about 18 wt. %, or about 20 wt. %. In some embodiments, metformin is present at a concentration of about 1 mg/mL, about 5 mg/mL, about 10 mg/mL, about 15 mg/mL, about 20 mg/mL, about 30 mg/mL, about 40 mg/mL, about 50 mg/mL, about 60 mg/mL, about 70 mg/mL, about 80 mg/mL, about 90 mg/mL, or about 100 mg/mL.

In some embodiments, sodium ascorbate is present in the pharmaceutical composition at an amount from about 2 wt. % to about 20 wt. %, from about 2 wt. % to about 10 wt. %, from about 2 wt. % to about 8 wt. %, from about 2 wt. % to about 6 wt. %, or from about 2 wt. % to about 4 wt. % of the composition. Sodium ascorbate can be present at about 2 wt. %, about 3 wt. %, about 4 wt. %, about 5 wt. %, about 6 wt. %, about 7 wt. %, about 8 wt. %, about 9 wt. %, about 10 wt. %, about 12 wt. %, about 14 wt. %, about 16 wt. %, about 18 wt. %, or about 20 wt. %. In some embodiments, sodium ascorbate is present at a concentration of about 0.1 mM, about 0.2 mM, about 0.3 mM, about 0.4 mM, about 0.5 mM, about 1 mM, about 5 mM, about 10 mM, about 15 mM, about 20 mM, about 30 mM, about 40 mM, about 50 mM, about 60 mM, about 70 mM, about 80 mM, about 90 mM, or about 100 mM.

In some embodiments, the pharmaceutical composition further comprises water. In some embodiments, the pharmaceutical composition can be an anhydrous pharmaceutical composition.

In some embodiments, the pharmaceutical composition further comprises at least one additional component, for example, a combination therapy. In some examples, the pharmaceutical composition further comprises at least one anti-inflammatory component such as a corticosteroid.

In certain embodiments, the compounds of the present disclosure are included in a pharmaceutical composition comprising one or more pharmaceutically-acceptable excipients, carriers, stabilizers, dispersing agents, suspending agents, thickening agents, and/or diluents. The pharmaceutical composition can facilitate administration of the compound to an organism. Pharmaceutical compositions can be administered in therapeutically-effective amounts as pharmaceutical compositions by various forms and routes including, for example, intravenous, subcutaneous, intramuscular, inhalation, oral, parenteral, ophthalmic, otic, subcutaneous, transdermal, nasal, intravitreal, intratracheal, intrapulmonary, transmucosal, vaginal, and topical administration.

In some embodiments, the pharmaceutical composition further comprises one or more pharmaceutically-acceptable excipients or carriers. Non-limiting examples of pharmaceutically-acceptable excipients include fillers/vehicles, solvents/co-solvents, preservatives, antioxidants, suspending agents, surfactants, antifoaming agents, buffering agents, chelating agents, sweeteners, flavoring agents, binders, extenders, disintegrants, diluents, lubricants, fillers, wetting agents, glidants, and combinations thereof. Non-limiting examples of pharmaceutically-acceptable carriers include saline solution, Ringer's solution, and dextrose solution. Further carriers include sustained release preparations such as semipermeable matrices of solid hydrophobic polymers containing the compound disclosed herein, wherein the matrices are in the form of shaped articles, such as films, liposomes, microparticles, and microcapsules.

In some embodiments, the pharmaceutical composition further comprises one or more fillers. Non-limiting examples of fillers include cellulose and cellulose derivatives such as microcrystalline cellulose; starches such as dry starch, hydrolyzed starch, and starch derivatives such as corn starch; cyclodextrin; sugars such as powdered sugar and sugar alcohols such as lactose, mannitol, sucrose and sorbitol; inorganic fillers such as aluminum hydroxide gel, precipitated calcium carbonate, carbonate, magnesium aluminometasilicate, dibasic calcium phosphate; and sodium chloride, silicon dioxide, titanium dioxide, titanium oxide, dicalcium phosphate dihydrate, calcium sulfate, alumina, kaolin, talc, and combinations thereof. Fillers can be present in the composition from about 20 wt % to about 65 wt %, about 20 wt % to about 50 wt %, about 20 wt % to about 40 wt %, about 45 wt % to about 65 wt %, about 50 wt % to about 65 wt %, or about 55 wt % to about 65 wt % of the total weight of the composition.

In some embodiments, the pharmaceutical composition further comprises one or more disintegrants. Non-limiting examples of disintegrants include starches, alginic acid, crosslinked polymers such as crosslinked polyvinylpyrrolidone, croscarmellose sodium, potassium starch glycolate, sodium starch glycolate, clays, celluloses, starches, gums, and combinations thereof. Disintegrants can be present in the composition from about 1 wt % to about 10 wt %, about 1 wt % to about 9 wt %, about 1 wt % to about 8 wt %, about 1 wt % to about 7 wt %, about 1 wt % to about 6 wt %, or about 1 wt % to about 5 wt % of the total weight of the composition.

In some embodiments, the pharmaceutical composition further comprises one or more binders, for example, celluloses such as hydroxypropylcellulose, methyl cellulose, and hydroxypropylmethylcellulose; starches such as corn starch, pregelatinized starch, and hydroxpropyl starch; waxes and natural and synthetic gums such as acacia, tragacanth, sodium alginate; synthetic polymers such as polymethacrylates and polyvinylpyrrolidone; and povidone, dextrin, pullulane, agar, gelatin, tragacanth, macrogol, or combinations thereof. Binders can be present in the composition from about 0.5 wt % to about 5 wt %, about 0.5 wt % to about 4 wt %, about 0.5 wt % to about 3 wt %, about 0.5 wt % to about 2 wt %, or about 0.5 wt % to about 1 wt % of the total weight of the composition.

In some embodiments, the pharmaceutical composition further comprises one or more wetting agents, for example, oleic acid, glyceryl monostearate, sorbitan mono-oleate, sorbitan monolaurate, triethanolamine oleate, polyoxyethylene sorbitan mono-oleate, polyoxyethylene sorbitan monolaurate, sodium oleate, sodium lauryl sulfate, poloxamers, poloxamer 188, polyoxyethylene ethers, polyoxyethylene sorbitan fatty acid esters, polyoxyethylene fatty acid esters, polyethylene glycol fatty acid esters, polyoxyethylene hardened castor oil, polyoxyethylene alkyl ethers, polysorbates, cetyl alcohol, glycerol fatty acid esters (for example, triacetin, glycerol monostearate, etc.), polyoxymethylene stearate, sodium lauryl sulfate, sorbitan fatty acid esters, sucrose fatty acid esters, benzalkonium chloride, polyethoxylated castor oil, or a combination thereof. Wetting agents can be present in the composition from about 0.1 wt % to about 1 wt %, about 0.1 wt % to about 2 wt %, about 0.1 wt % to about 3 wt %, about 0.1 wt % to about 4 wt %, or about 0.1 wt % to about 5 wt % of the total weight of the composition.

In some embodiments, the pharmaceutical composition further comprises one or more lubricants. Non-limiting examples of lubricants include magnesium stearate, calcium hydroxide, talc, corn starch, sodium stearyl fumarate, alkali-metal and alkaline earth metal salts, waxes, boric acid, sodium benzoate, sodium acetate, sodium chloride, leucine, polyethylene glycol (PEG), a methoxypolyethylene glycol, propylene glycol, sodium oleate, glyceryl behenate, glyceryl palmitostearate, glyceryl benzoate, magnesium lauryl sulfate, sodium lauryl sulfate, and combinations thereof. Lubricants can be present in the composition from about 0.1 wt % to about 5 wt %, about 0.1 wt % to about 4 wt %, about 0.1 wt % to about 3 wt %, about 0.1 wt % to about 2 wt %, or about 0.1 wt % to about 1 wt % of the total weight of the composition.

In some embodiments, the pharmaceutical composition further comprises one or more glidants. Non-limiting examples of glidants include colloidal silicon dioxide, talc, sodium lauryl sulfate, native starch, and combinations thereof. Glidants can be present in the composition from about 0.05 wt % to about 1 wt %, about 0.05 wt % to about 0.9 wt %, about 0.05 wt % to about 0.8 wt %, about 0.05 wt % to about 0.5 wt %, or about 0.05 wt % to about 0.1 wt % of the total weight of the composition.

In some embodiments, the pharmaceutical composition further comprises one or more preservative agents. Non-limiting examples of preservative agents include sodium benzoate, paraoxybenzoic acid esters, methyl, ethyl, butyl, and propyl parabens, chlorobutanol, benzyl alcohol, phenylethylalcohol, dehydroacetic acid, sorbic acid, benzalkonium chloride (BKC), benzethonium chloride, phenol, phenylmercuric nitrate, thimerosal, and combinations thereof. Preservative agents can be included in the liquid dosage form. The preservative agents can be present in an amount sufficient to extend the shelf-life or storage stability, or both, of the liquid dosage form. Preservatives can be present in the composition from about 0.05 wt % to about 1 wt %, about 0.05 wt % to about 0.9 wt %, about 0.05 wt % to about 0.8 wt %, about 0.05 wt % to about 0.5 wt %, or about 0.05 wt % to about 0.1 wt % of the total weight of the composition.

In some embodiments, the pharmaceutical composition further comprises one or more flavoring agents. Non-limiting examples of flavoring agents include synthetic flavor oils and flavoring aromatics and/or natural oils, extracts from plants leaves, flowers, fruits, and combinations thereof. Additional examples include cinnamon oil, oil of wintergreen, peppermint oils, clove oil, bay oil, anise oil, eucalyptus, thyme oil, cedar leaf oil, oil of nutmeg, oil of sage, oil of bitter almonds, vanilla, citrus oil, including lemon, orange, grape, lime, grapefruit, and fruit essences, including apple, banana, pear, peach, strawberry, raspberry, cherry, plum, pineapple, apricot, strawberry flavor, tutti-fruity flavor, mint flavor, and combinations thereof. Flavoring agents can be present in the composition from about 0.1 wt % to about 5 wt %, about 0.1 wt % to about 4 wt %, about 0.1 wt % to about 3 wt %, about 0.1 wt % to about 2 wt %, or about 0.1 wt % to about 1 wt % of the total weight of the composition.

In some embodiments, the pharmaceutical composition further comprises one or more antioxidants. Non-limiting examples of antioxidants include flavonoids, anthocyanidins, anthocyanins, proanthocyanidins, and combinations thereof. Antioxidants can be present in the composition from about 0.05 wt % to about 1 wt %, about 0.05 wt % to about 0.9 wt %, about 0.05 wt % to about 0.8 wt %, about 0.05 wt % to about 0.5 wt %, or about 0.05 wt % to about 0.1 wt % of the total weight of the composition.

### Physical Form of the Pharmaceutical or Cosmetic Compositions

The pharmaceutical compositions can be in any physical form suitable for use in treating a subject. These forms can be referred to as a unit dosage form, such as an individual pill or tablet. In some embodiments, the pharmaceutical compositions can be formulated as liquids, lotions, creams, gels, ointments, tablets, capsules, granules, powders, suspensions, syrups, slurries, suppositories, patches, nasal sprays, aerosols, injectables, implantable sustained-release formulations, or mucoadherent films. In some embodiments, the pharmaceutical composition is formed as a tablet, a bi-layer tablet, a capsule, a multiparticulate, a drug coated sphere, a matrix tablet, or a multicore tablet. A physical form can be selected according to the desired method of treatment. In some embodiments, the physical form is a liquid, for example for oral, intravenous (IV), intraperitoneal (IP), or intramuscular (IM) administration. In some embodiments, the pharmaceutical composition is coated on a stent.

Pharmaceutical compositions can be manufactured by various methods such as mixing, dissolving, granulating, dragee-making, levigating, emulsifying, encapsulating, entrapping, or lyophilizing processes. Pharmaceutical compositions can be formulated using one or more physiologically acceptable carriers, diluents, excipients, or auxiliaries that facilitate processing of the active agent into preparations that can be used pharmaceutically. Proper formulation can be selected upon the route of administration chosen.

For topical administration the pharmaceutical compositions described herein can be formulated, for example, as solutions, gels, ointments, creams, and suspensions. Non-limiting examples of systemic compositions include those designed for administration by injection, for example, subcutaneous, intravenous injection (IV), intramuscular injection (IM), intraperitoneal injection (IP), and those designed for transdermal, subcutaneous, transmucosal oral, or pulmonary administration. For injection, the pharmaceutical compositions can be formulated in aqueous solutions, preferably in physiologically compatible buffers such as Hanks solution, Ringer's solution, or physiological saline buffer and/or in certain emulsion formulations. The solution can contain one or more formulatory agents such as suspending, stabilizing and/or dispersing agents. In some embodiments, the pharmaceutical compositions are provided in powder form for constitution with a suitable vehicle, for example, sterile pyrogen-free water, before use. For transmucosal administration, one or more penetrants appropriate to the barrier to be permeated can be used in the formulation.

Topical administration can be in the form of a cream. In some embodiments, topical administration is in the same body area where a moisturizing agent, such as ferulic acid or vitamin D, has been applied. In some embodiments, topical administration neutralizes or reverses the effects of an agent (such as hyaluronic acid or vitamin D₃) that blocks elastin production.

For oral liquid preparations such as, suspensions, elixirs, and solutions, suitable carriers, excipients, or diluents including water, glycols, oils, alcohols, etc. can be added. Flavoring agents, preservatives, and coloring agents can be added. For buccal administration, the compositions can be tablets, lozenges, etc.

For administration by inhalation, the pharmaceutical compositions can be delivered in the form of an aerosol spray from pressurized packs or a nebulizer, with the use of a suitable propellant, for example, dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, for example, gelatin for use in an inhaler or insufflator can be formulated containing a powder mix of the compound and a suitable powder base such as lactose or starch.

In some embodiments, the pharmaceutical compositions are immediate release pharmaceutical compositions, modified release pharmaceutical compositions, or a combination thereof. In some embodiments, the immediate release pharmaceutical composition releases the composition within a short period of time after administration, typically less than about 4 hours, less than about 3.5 hours, less than about 3 hours, less than about 2.5 hours, less than about 2 hours, less than about 90 minutes, less than about 60 minutes, less than about 45 minutes, less than about 30 minutes, less than about 20 minutes, or less than about 10 minutes.

In some embodiments, the modified release composition releases the tannic acid or ellagic acid at a sustained or controlled rate over an extended period of time or releases after a lag time after administration. For example, the tannic acid or ellagic acid can be released from the composition 4 hours after administration, 8 hours after administration, 12 hours after administration, 16 hours after administration, or 24 hours after administration. Modified release compositions can include extended release, sustained release, and delayed release compositions. In some embodiments, the modified release compositions release about 10% in about 2 hours, about 20% in 2 hours, about 40% in about 2 hours, about 50% in about 2 hours, about 10% in about 3 hours, about 20% in 3 hours, about 40% in about 3 hours, about 50% in about 3 hours, about 10% in about 4 hours, about 20% in 4 hours, about 40% in about 4 hours, about 50% in about 4 hours, about 10% in about 6 hours, about 20% in 6 hours, about 40% in about 6 hours, or about 50% in about 6 hours.

In some embodiments, modified release compositions comprise a matrix selected from microcrystalline cellulose, sodium carboxymethylcellulose, hydroxyalkylcelluloses such as hydroxy propyl methylcellulose and hydroxypropylcellulose, polyethylene oxide, alkylcelluloses such as methylcellulose and ethylcellulose, polyethylene glycol, polyvinylpyrrolidone, cellulose acetate, cellulose acetate butyrate, cellulose acetate phthalate, cellulose acetate trimellitate, polyvinyl acetate phthalate, polyalkylmethacrylates, polyvinyl acetate, and mixtures thereof.

The modified release compositions can also be formulated as a depot preparation. Such long-acting formulations can be administered by implantation (for example subcutaneously or intramuscularly) or by intramuscular injection. Thus, for example, the compounds can be formulated with suitable polymeric or hydrophobic materials, for example as an emulsion in an acceptable oil, or ion exchange resins, or as sparingly soluble derivatives, for example, as a sparingly soluble salt.

In some embodiments, the composition is a part of an injectable dermal filler treatment. Dermal filler treatments can be used to restore volume and fullness to the skin lost through the aging process. Non-limiting examples of hyaluronic acid-based filler treatments include Restylane® and Juvéderm®.

Treatments can be performed at any effective schedule. For example, the pharmaceutical compositions disclosed herein can be administered once, as needed, once daily, twice daily, three times a day, four times a day, once a week, twice a week, three times a week, four times a week, five times a week, six times a week, seven times a week, every other week, or every other day for one or more dosing cycles. A dosing cycle can include administration for about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, or longer. After a first cycle, a subsequent cycle can begin, for example, 1, 2, 3, 4, 5, 6, 7, 8, 9, 10, 11, or 12 weeks later. The treatment regime can include, for example, 1, 2, 3, 4, 5, or 6 cycles, each cycle being spaced apart by, for example, 1, 2, 3, 4, 5, 6, 7, 8, 9, 10, 11, or 12 weeks. The specific dose level and frequency of dosage for any subject can vary and depends on factors including the species, age, body weight, general health, gender, and diet of the subject, the mode and time of administration, rate of excretion, drug combination, and severity of the particular condition.

### Methods of Treatment

Methods described herein are directed to increasing elastin concentration in a subject, the method comprising administering a pharmaceutical composition to the subject, wherein the pharmaceutical composition comprises a therapeutically-effective amount of a first agent and a therapeutically-effective amount of a second agent, wherein the first agent is tannic acid, ellagic acid, or a combination thereof, and the second agent is vitamin D, hyaluronic acid, ferulic acid, or a combination thereof. In some embodiments, the concentration of elastin in the extracellular matrix increases by about 100%-500% after administering the pharmaceutical composition to the subject.

In some embodiments, a method of treating wrinkles on a subject's skin comprises administering a pharmaceutical composition to the subject, wherein the pharmaceutical composition comprises a therapeutically-effective amount of a first agent and a therapeutically-effective amount of a second agent, wherein the first agent is tannic acid, ellagic acid, or a combination thereof, and the second agent is vitamin D, hyaluronic acid, ferulic acid, or a combination thereof.

In some embodiments, a method of treating heart failure comprises administering a pharmaceutical composition to the subject, wherein the pharmaceutical composition comprises a therapeutically-effective amount of a first agent and a therapeutically-effective amount of a second agent, wherein the first agent is tannic acid, ellagic acid, or a combination thereof, and the second agent is vitamin D, hyaluronic acid, ferulic acid, or a combination thereof.

In some embodiments, a method of treating hypertension comprises administering a pharmaceutical composition to the subject, wherein the pharmaceutical composition comprises a therapeutically-effective amount of a first agent and a therapeutically-effective amount of a second agent, wherein the first agent is tannic acid, ellagic acid, or a combination thereof, and the second agent is vitamin D, hyaluronic acid, ferulic acid, or a combination thereof.

In some embodiments, a method of treating a wound comprises administering a pharmaceutical composition to the subject, wherein the pharmaceutical composition comprises a therapeutically-effective amount of a first agent and a therapeutically-effective amount of a second agent, wherein the first agent is tannic acid, ellagic acid, or a combination thereof, and the second agent is vitamin D, hyaluronic acid, ferulic acid, or a combination thereof.

In some embodiments, a method of protecting a subject from a viral infection comprises administering a pharmaceutical composition to the subject, wherein the pharmaceutical composition comprises a therapeutically-effective amount of a first agent and a therapeutically-effective amount of a second agent, wherein the first agent is tannic acid, ellagic acid, or a combination thereof, and the second agent is vitamin D, hyaluronic acid, ferulic acid, or a combination thereof.

The compounds and pharmaceutical compositions described herein can be administered at prophylactically-effective dosage levels to mitigate or reduce a likelihood of occurrence of the recited conditions, disorders, and diseases.

Administration can be performed by any suitable method. Non-limiting examples of delivery methods include topical delivery, subcutaneous delivery, intravenous injection (IV) delivery, intramuscular injection (IM) delivery, intraperitoneal injection (IP) delivery, transdermal delivery, subcutaneous delivery, oral delivery, transmucosal oral delivery, pulmonary delivery, inhalation delivery, intranasal delivery, buccal delivery, rectal delivery, vaginal delivery, coated on a stent, and combinations thereof. In some embodiments, the administering comprises topical delivery. In some examples, the administering comprises subcutaneous delivery.

### Kits

In some embodiments, kits are provided for treating or reducing a likelihood of viral infection in a subject. The kit can comprise a first container containing a pharmaceutical composition, wherein the pharmaceutical composition comprises an effective amount of a first agent and an effective amount of a second agent, wherein the first agent is tannic acid or ellagic acid or a combination thereof, and the second agent is vitamin D, hyaluronic acid, ferulic acid, or a combination thereof; and instructions for the administration of the pharmaceutical composition to the subject. Any of the above-described pharmaceutical compositions can be included in the kit. The kit can further comprise a second container, a third container, and so on containing additional pharmaceutical compositions or other active ingredients. In some embodiments, the first container can contain a pharmaceutical composition, and the second container can contain a solvent to be mixed with the pharmaceutical composition before administering to the subject according to the instructions.

In some embodiments, the kit can comprise a first container containing a pharmaceutical composition, wherein the pharmaceutical composition comprises an effective amount of a first agent and a second agent or a combination thereof, wherein the first agent is a polyphenolic acid, and the second compound is vitamin D, hyaluronic acid, ferulic acid, or a combination thereof; and instructions for the administration of the pharmaceutical composition to the subject. The polyphenolic compound can be tannic acid or ellagic acid. Any of the above-described pharmaceutical compositions can be included in the kit. The kit can further comprise a second container, a third container, and so on containing additional pharmaceutical compositions or other active ingredients. In some examples, the first container can contain a pharmaceutical composition, and the second container can contain a solvent to be mixed with the pharmaceutical composition before administering to the subject according to the instructions.

In some embodiments, the kit comprises an ampoule, disposable syringe, capsule, vial, or tube. In some embodiment, the kit comprises a single dose container or multiple dose containers, each containing the compositions of embodiments herein. In some embodiments, each dose container contains one or more unit doses. In some embodiments, the kit includes an applicator. In some embodiments, the kit includes the composition in a gel or ointment, for example, in a tube. In some embodiments, the composition is provided in a viscous liquid (such as carboxylmethylcellulose, hydroxypropylmethycellulose, polyethylene glycol, glycerin, polyvinyl alcohol, or oil containing drops) for rubbing into the skin. In some embodiments, the compositions have preservatives or be preservative-free (for example, in a single-use container).

### EXAMPLES

### Example 1: Preparation of Fibroblasts

Biological effects of tannic acid in the presence of hyaluronic acid or ferulic acid were tested on dermal fibroblasts derived from punch biopsies of healthy skin. All fibroblasts were isolated by allowing migration out of skin explants and passaged by trypsinization. Fibroblasts were maintained in alpha-minimum essential medium supplemented with 20 mM HEPES, 1% antibiotics and antimycotics, 1% L-Glutamate and 2% fetal bovine serum (FBS). Cells were plated (50×10⁵ cells/dish) to reach confluency and were cultured for 7 days in the presence or absence of hyaluronic acid (0.1%), ferulic acid (5 μg/mL), tannic acid (2 μg/mL), or a combination thereof.

### Example 2: Immunohistochemistry

Cell cultures prepared according to Example 1 maintained in the presence or absence of tannic acid, hyaluronic acid, or ferulic acid were fixed in cold 100% methanol at 20° C. for detection of elastin or in 4% paraformaldehyde at room temperature for detection of collagen I. The multiple parallel cultures were incubated with 10 mg/mL of polyclonal antibody to tropoelastin (Elastin Products, Owensville, MI), or polyclonal antibody to collagen type I (Chemicon, Temecula, CA). Cultures were incubated with the respective fluorescein-conjugated goat anti-rabbit, goat anti-mouse, or rabbit anti-goat secondary antibodies. Nuclei were counter stained with propidium iodide (Sigma, St. Louis, MO). All cultures were examined with a Nikon Eclipse E1000 microscope attached to a cooled CCD camera (QImaging, Retiga EX) and analyzed with the computer-generated morphometric analysis system, in which the Image-Pro Plus software (Media Cybernetics, Silver Springs, MD) estimates the proportion of areas marked with green fluorescence, in relation to the entire (1 square mm) analyzed field.

### Example 3: Effects of Low and High Concentrations of Tannic Acid on Elastin Concentration

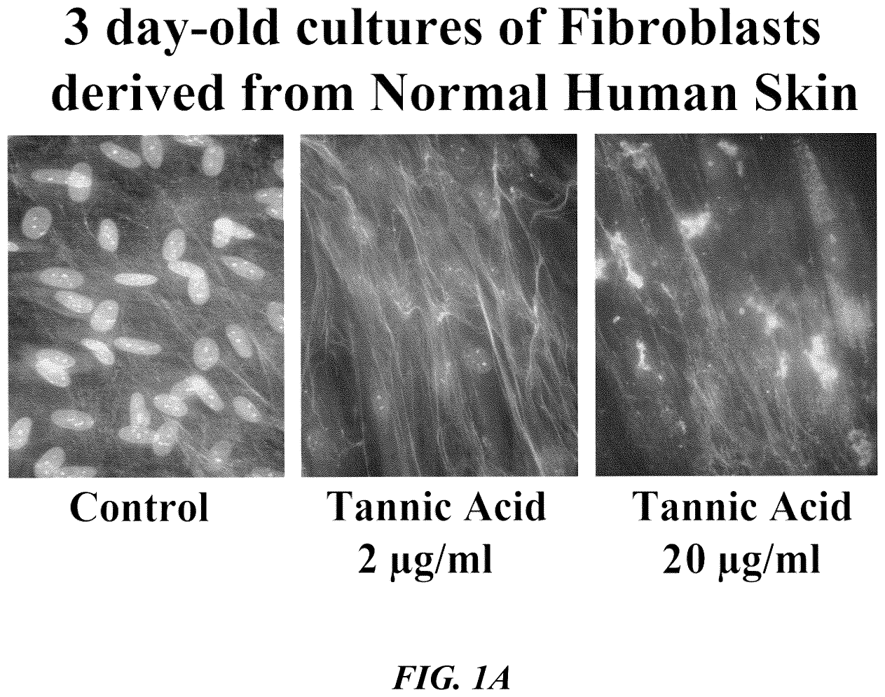

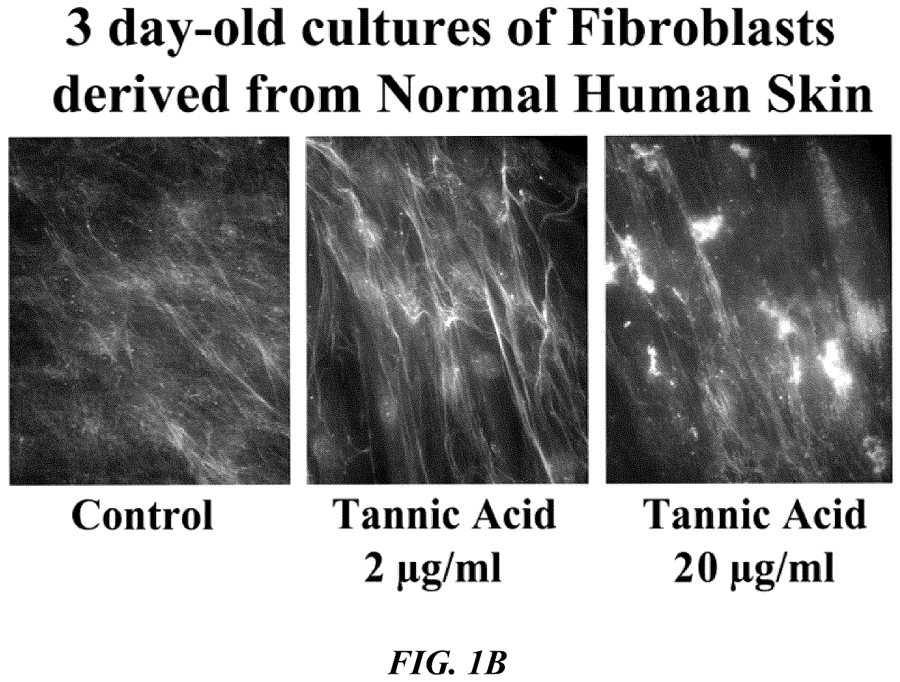

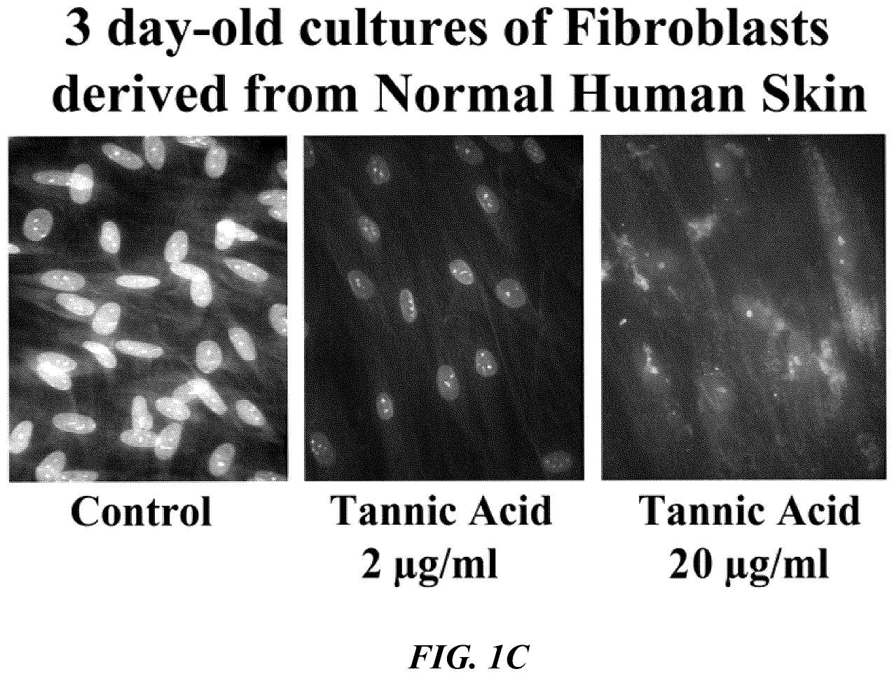

Cultured human dermal fibroblasts were grown in the presence or absence of tannic acid at low concentration (2 μg/mL) and high concentration (20 μg/mL). Immunodetection of elastin was performed as described in Example 2. Results are shown in FIGS. 1A-1C. Low concentrations of tannic acid led to improved elastin concentration as compared to high concentrations of tannic acid, which caused elastosis.

### Example 4: Effects of Tannic Acid on Elastin Concentration in Presence of Vitamin D

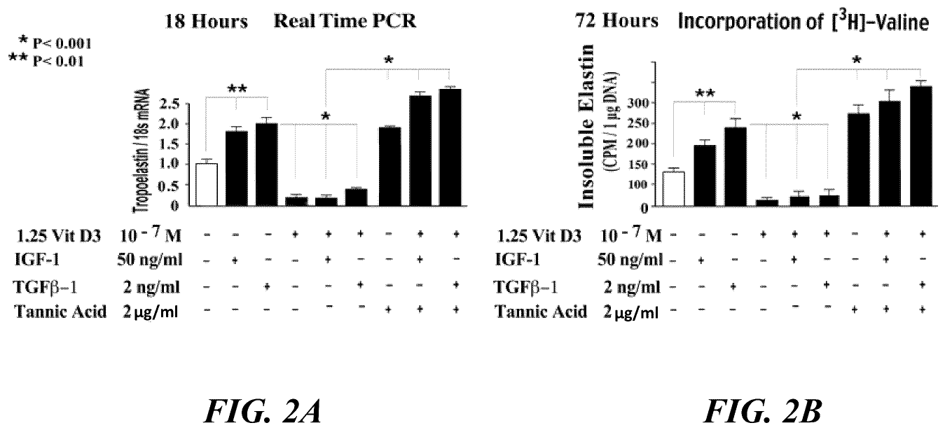

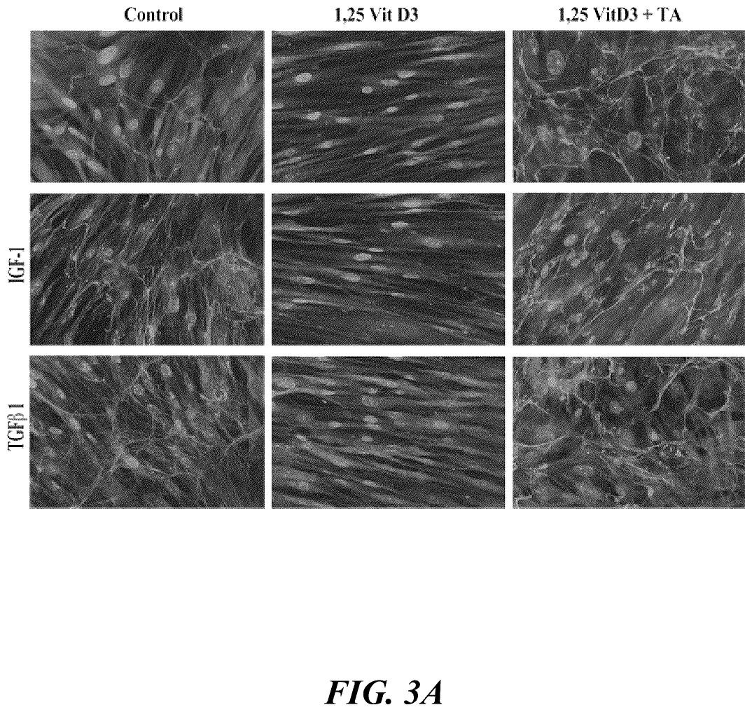

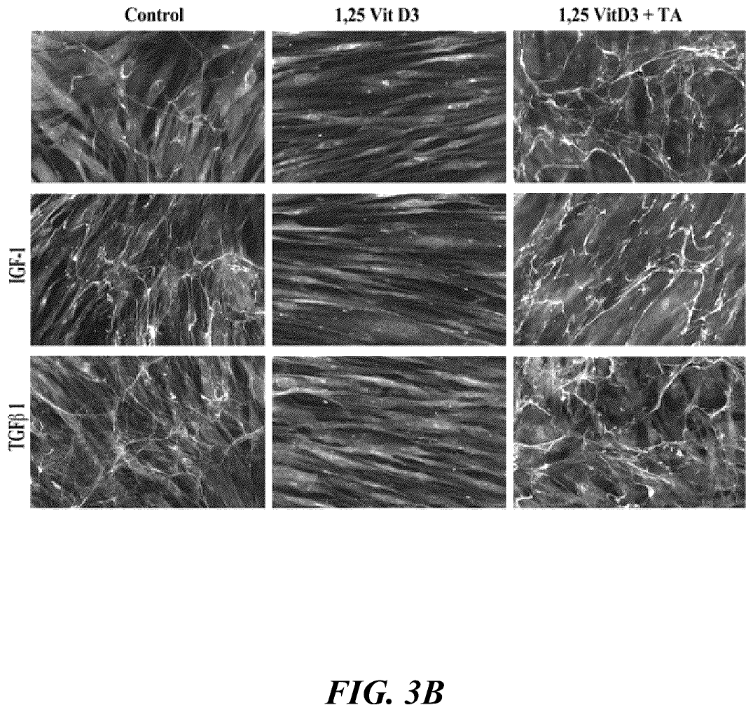

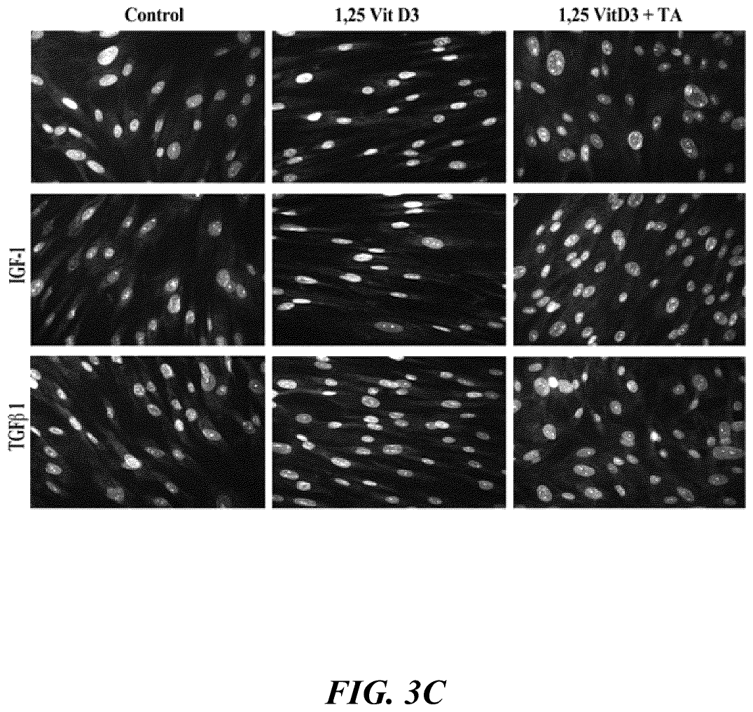

This example shows effects of tannic acid and vitamin D on concentrations of elastin. Cultured human dermal fibroblasts were grown in the presence of or absence of vitamin D (10⁻⁷ M), tannic acid (2 ng/mL), or a combination of vitamin D and tannic acid (2 μg/mL) (FIGS. 2A-2B and FIGS. 3A-3C). Immunodetection of elastin was performed as described above.

While vitamin D causes inhibition of elastin production, as described previously, the addition of tannic acid abolished the inhibitory effect of vitamin D on elastin fiber formation, and additionally caused an improvement in net deposition of elastin.

### Example 5: Effects of Tannic Acid on Elastin Concentration in Presence of Hyaluronic Acid

This example shows effects of tannic acid and hyaluronic acid on concentrations of elastin. Cultured human dermal fibroblasts were grown in the presence of or absence hyaluronic acid (0.1%), tannic acid (2 μg/mL), or a combination of hyaluronic acid (0.1%) and tannic acid (2 μg/mL). Immunodetection of elastin was performed as described above.

While hyaluronic acid causes inhibition of elastin production, as described previously, the addition of tannic acid abolished the inhibitory effect of hyaluronic acid on elastin fiber formation, and additionally caused an improvement in net deposition of elastin.

### Example 6: Effects of Tannic Acid on Collagen Concentration in Presence of Hyaluronic Acid

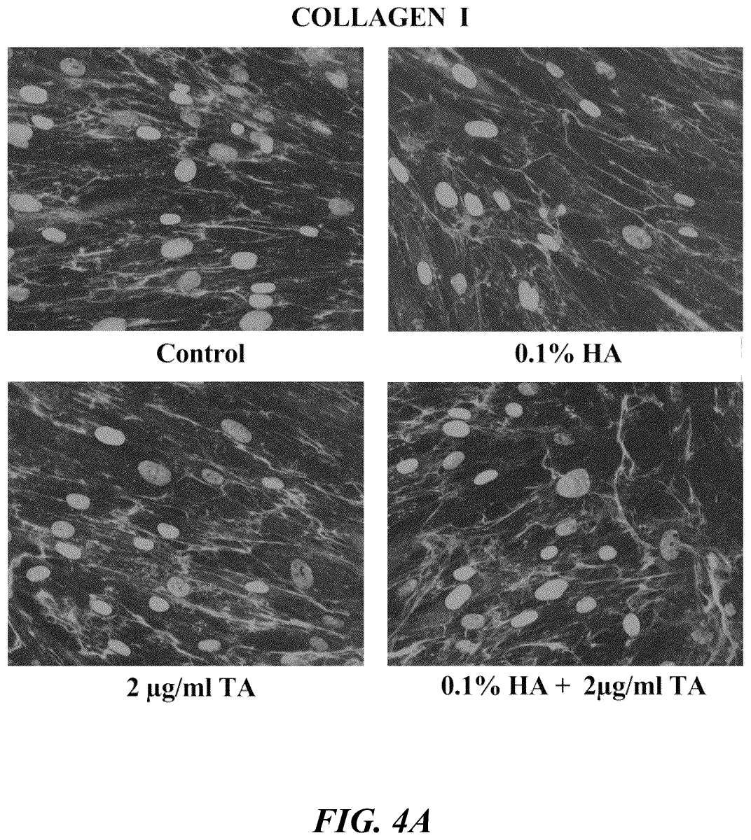

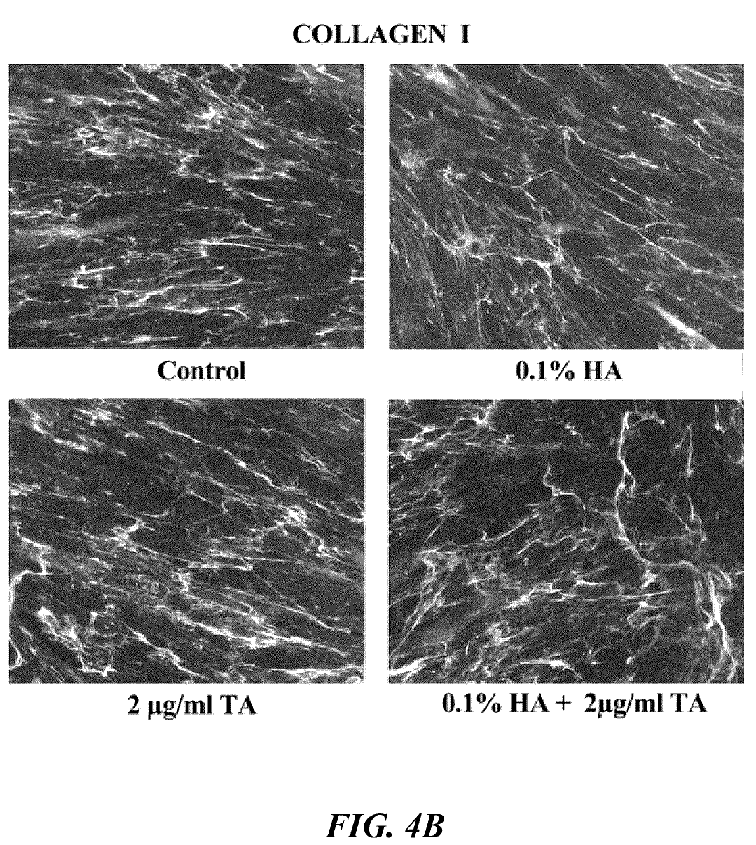

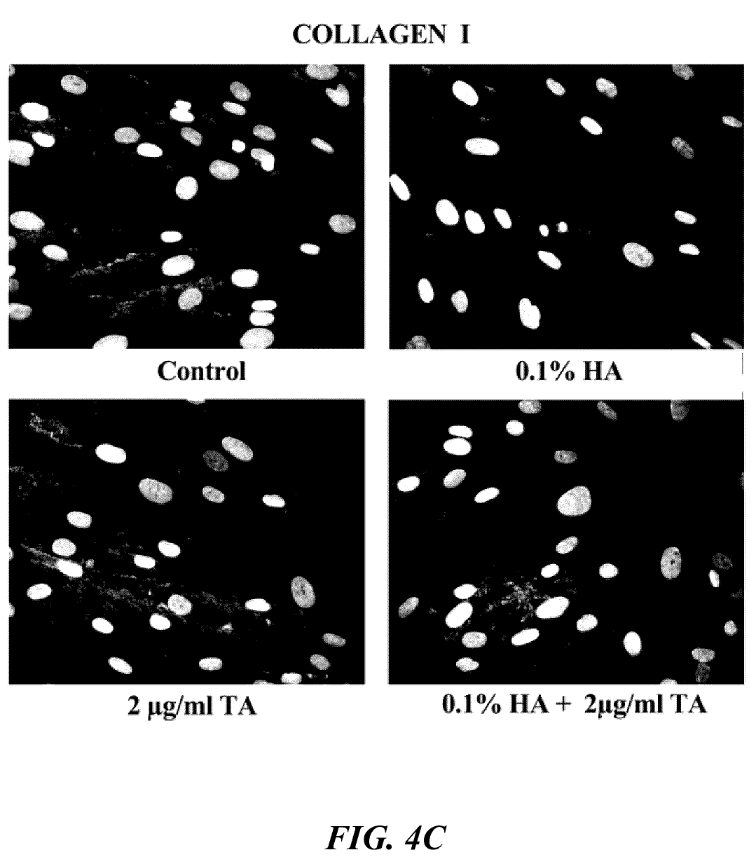

This example shows effects of tannic acid and hyaluronic acid on concentrations of collagen. Cultured human dermal fibroblasts were grown in the presence or absence of hyaluronic acid (0.1%), tannic acid (2 μg/mL), or a combination of hyaluronic acid (0.1%) and tannic acid (2 μg/mL). Immunodetection of collagen was performed as described above, and results are shown in FIGS. 4A-4C.

### Example 7: Effect of Tannic Acid on Concentrations of Elastin and Collagen in Presence of Ferulic Acid

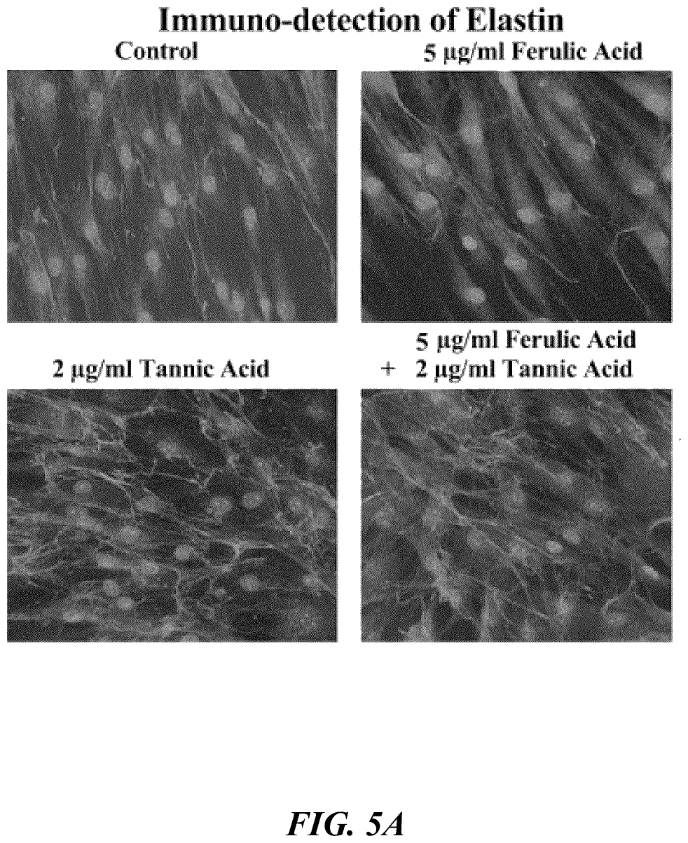

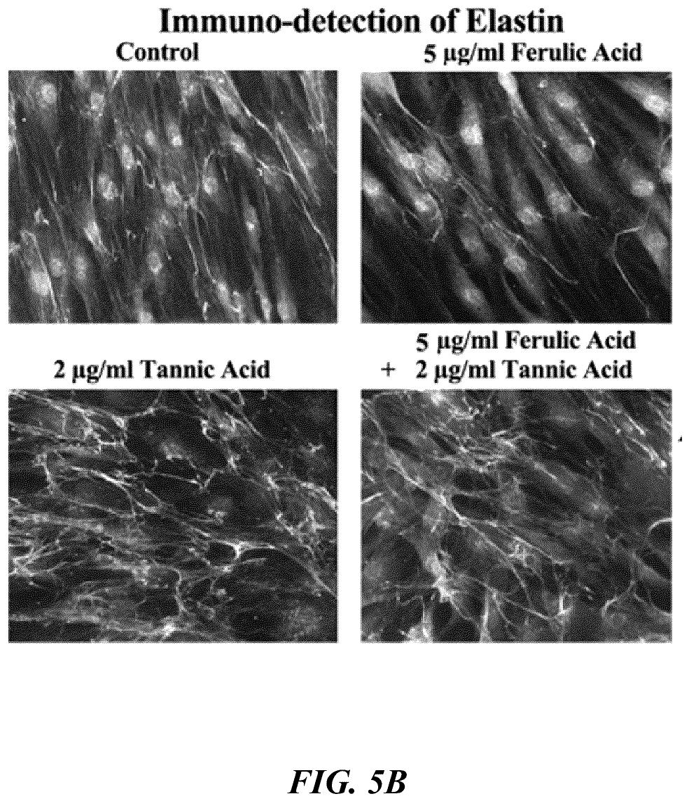

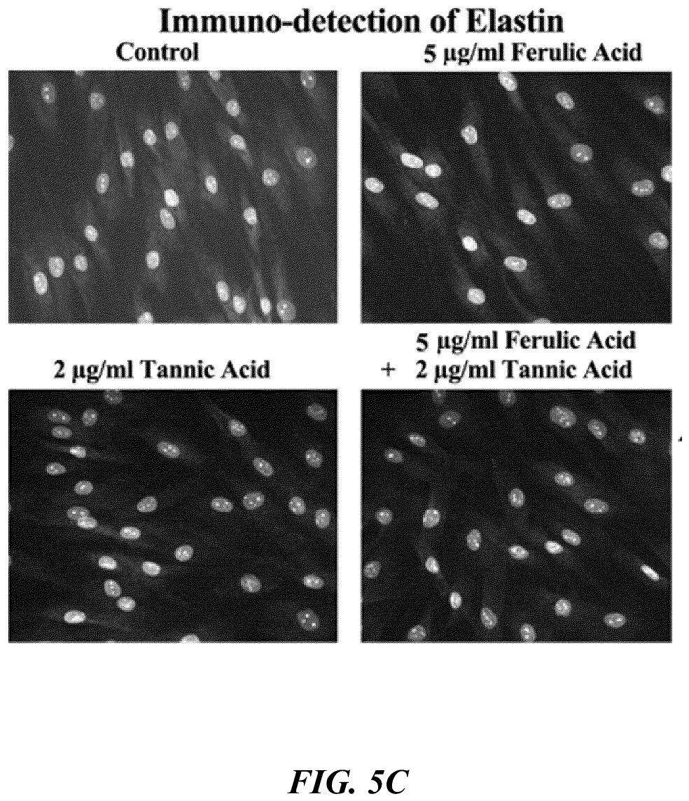

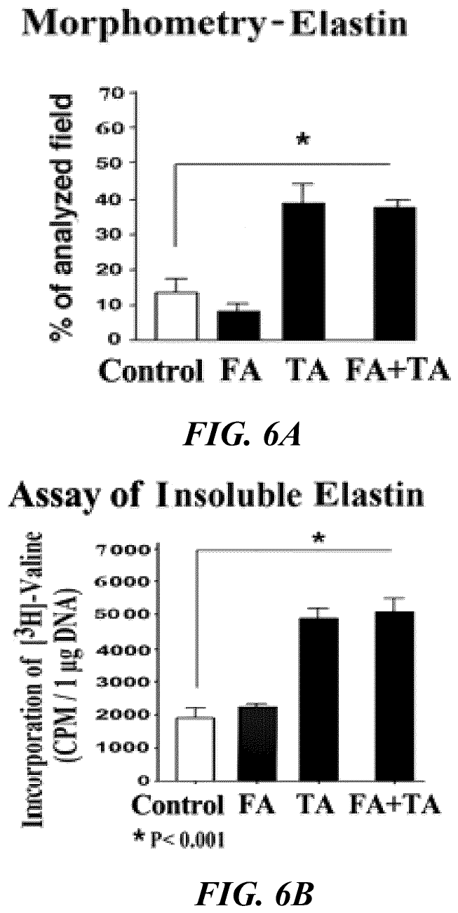

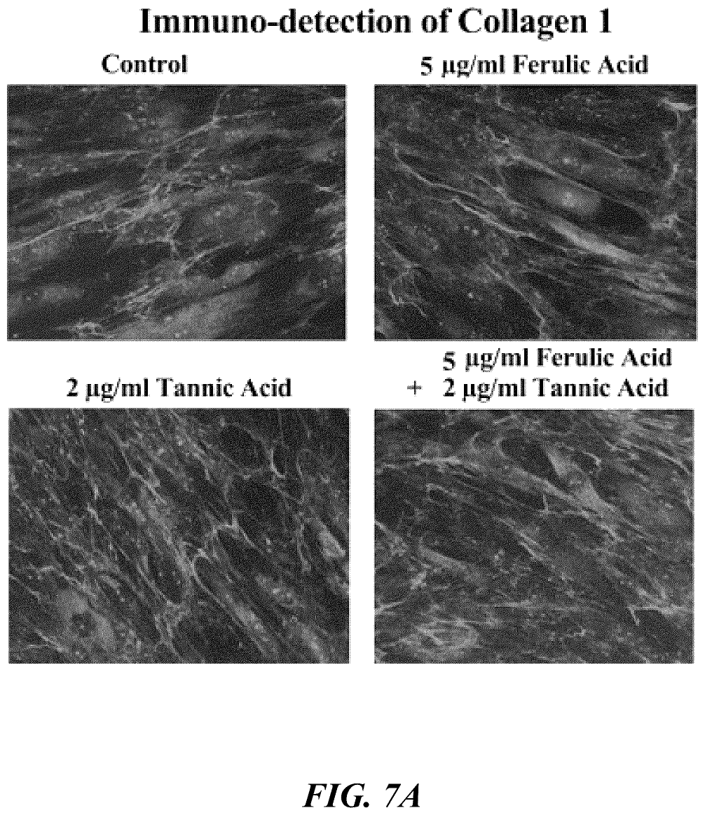

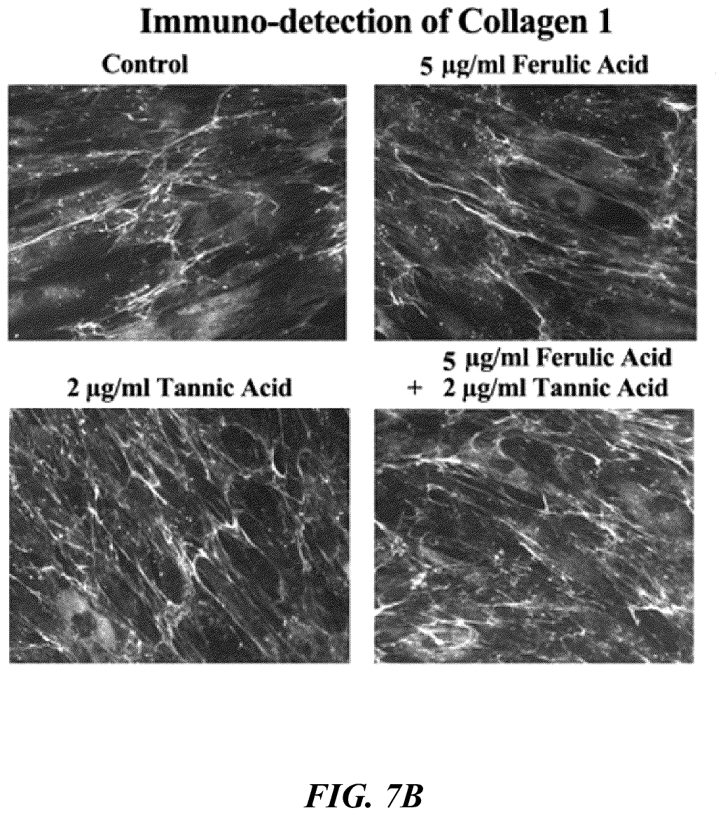

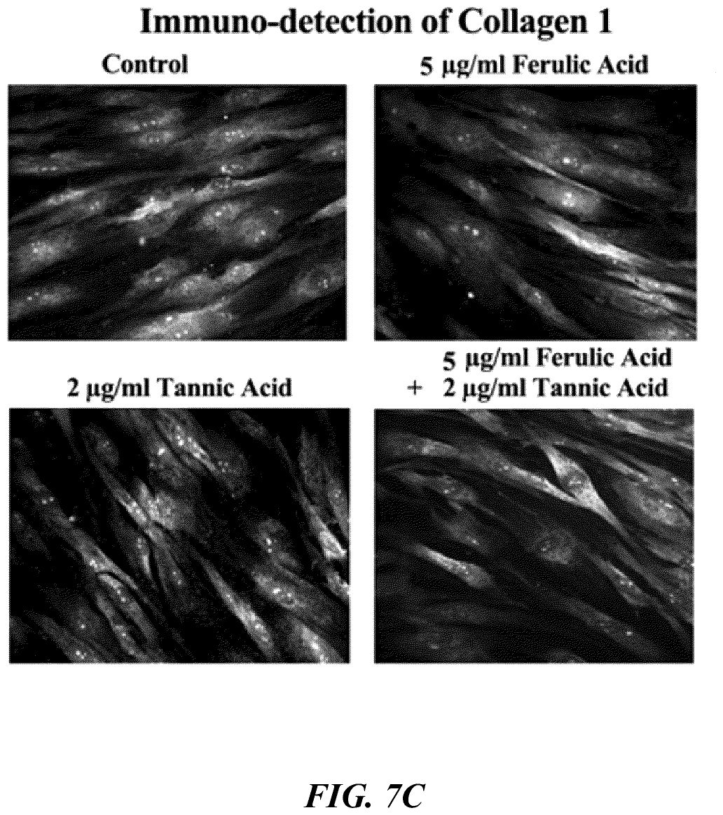

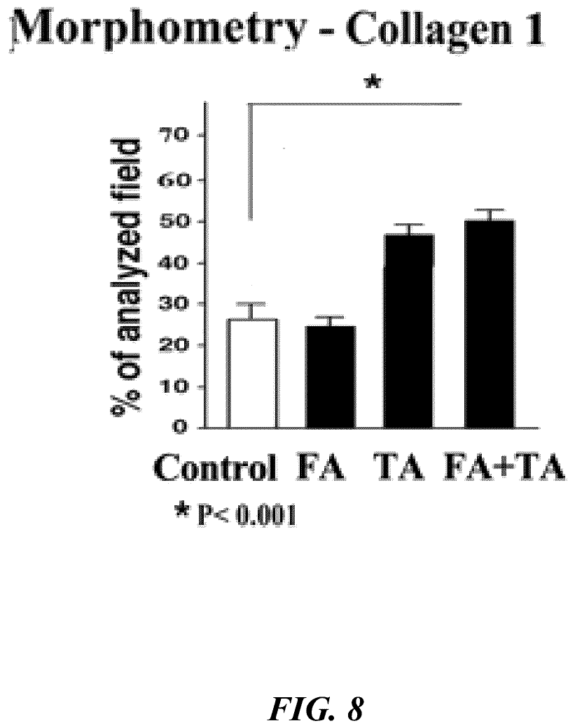

Cultured human dermal fibroblasts were grown in the presence or absence of ferulic acid (5 μg/mL), tannic acid (2 μg/mL), or a combination of ferulic acid (5 μg/mL) and tannic acid (2 μg/mL). Immunodetection of elastin and collagen I was performed as described above. Results are shown in FIGS. 5A-5C, and FIGS. 6A-6B for elastin and FIGS. 7A-7C and FIG. 8 for collagen.

Ferulic acid had a negative effect on concentrations of elastin, and minimal effect on concentrations of collagen. The addition of tannic acid at low concentrations (2 μg/mL) improved production of elastin even in the presence of ferulic acid, which otherwise had the effect of decreasing elastin amounts.

### Example 8: Effect of Tannic Acid on Concentration of Elastin in Presence of Ferulic Acid or Copper Sulfate

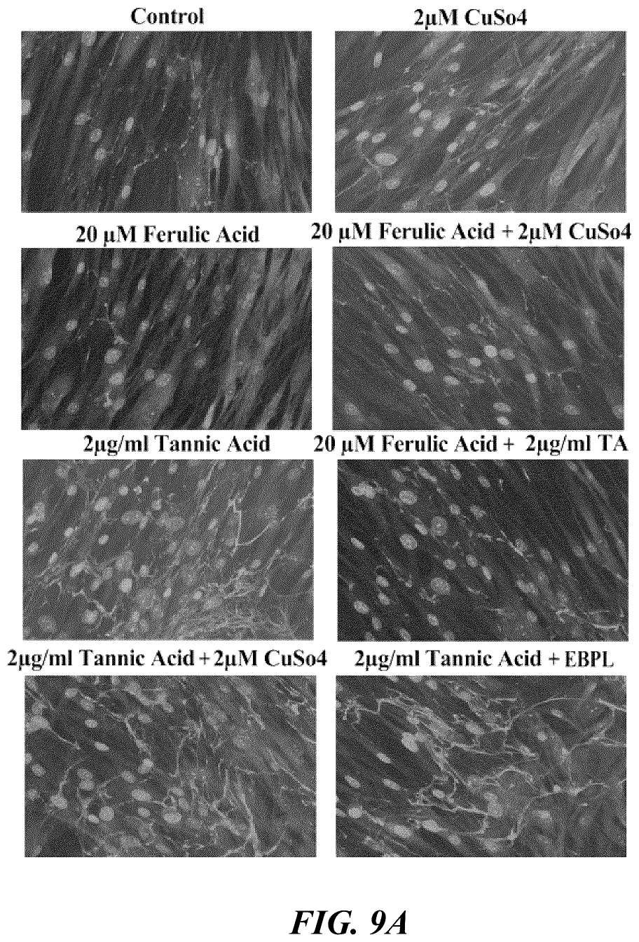

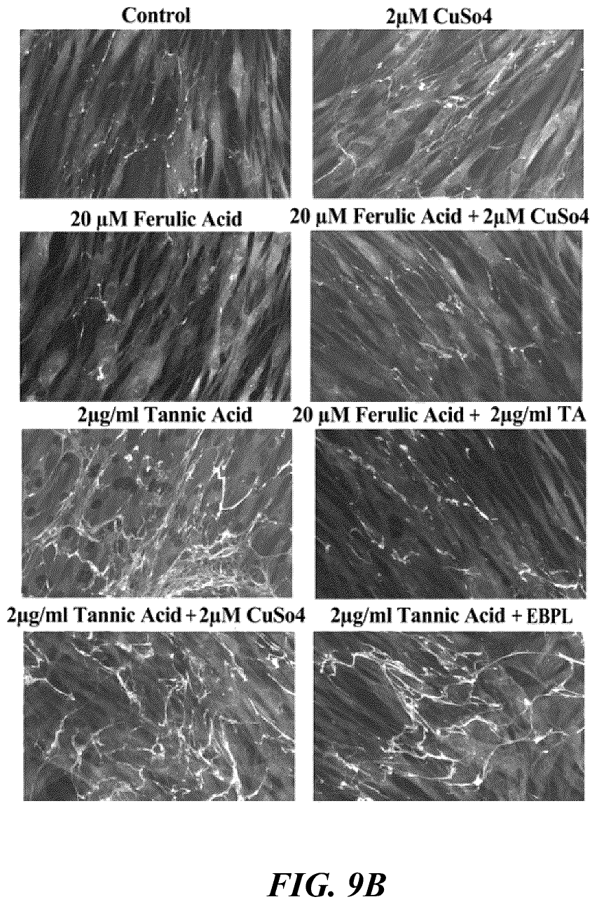

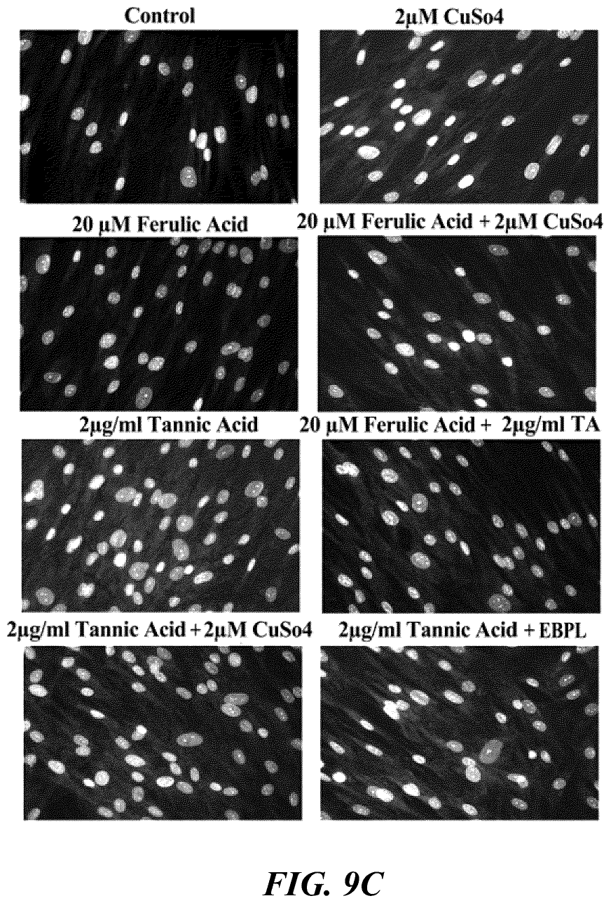

This example shows the contrasting effects of compounds such as copper sulfate, which has little effect on elastin, to compounds such as ferulic acid, which leads to a reduction in elastin. Cultured human dermal fibroblasts were grown in the presence or absence of ferulic acid (20 μM), tannic acid (2 μg/mL), or copper sulfate (2 μM). Fibroblasts were also grown in presence of a combination of ferulic acid (20 μM) and copper sulfate (2 μM), a combination of ferulic acid (20 μM) and tannic acid (2 μg/mL), and a combination of tannic acid (2 μg/mL) and copper sulfate (2 μM). Immunodetection of elastin was performed as described above. Results are shown in FIGS. 9A-9C.

### Example 9: Method of Treating Wrinkled Skin in a Subject in Need of Improved Elastin Concentration

In this example, an ointment containing tannic acid (2 μg/mL), hyaluronic acid (0.1%), ferulic acid (3.88 μg/mL), and containing one or more binders, lubricants, emulsifiers, or other pharmaceutically-acceptable excipients, was applied to the patient's skin twice daily for 4 weeks. Post-treatment results were compared to pre-treatment condition using high-quality color photographic images taken in the same lighting, contrast, and exposure. At the end of treatment, skin was noticeably smoother and less furrowed, creased. Results were verified by comparing amounts of shadows in the pre-treatment images to those of the post-treatment images using commercially-available color quantification software.

### Example 10: Method of Treating Skin Roughness in a Subject in Need of Improved Elastin Concentration

In this example, an ointment containing tannic acid (2 μg/mL), hyaluronic acid (0.1%), ferulic acid (3.88 μg/mL), and containing one or more binders, lubricants, emulsifiers, or other pharmaceutically acceptable excipients, is applied to the patient's skin twice daily for 4 weeks. Post-treatment results are compared to pre-treatment condition using high-quality color photographic images taken in the same lighting, contrast, and exposure. At the end of treatment, skin is noticeably smoother, with less roughness and more consistent color. Results are verified comparing amount of shadows in the pre-treatment to the post-treatment images using commercially-available color quantification software.

### Example 11: Method of Treating Arteriosclerosis in a Subject

In this example, a formulation for injection containing lactated ringer's injection, tannic acid (2 μg/mL), ferulic acid (3.88 μg/mL), and vitamin D3 (40 ng/mL) is administered to a patient with arteriosclerosis every day for a week. Alternatively, a stent is coated with a composition comprising tannic acid (2 μg/mL), ferulic acid (3.88 μg/mL), and vitamin D3 (40 ng/mL) and is inserted into an artery as necessary.

### Example 12: A Kit for Treating Sun-Damaged Skin

In this example, a kit for treating sun-damaged skin of a subject comprises a viscous liquid composed of glycerin (25%), cetearyl alcohol (2%), water, ellagic acid (2 μg/mL), hyaluronic acid (0.1%), and ferulic acid (3.88 μg/mL), packaged in a dispensing tube. Instructions for use are included in the kit package.

### Example 13: A Kit for Treating Ageing Skin in Need of Increased Elastin

In this example, a kit contains a dispensing container that is filled with a gel pharmaceutical formulation. The gel is composed of methylcellulose (1%), water, vitamin D (100 nM), hyaluronic acid (0.1%), and tannic acid (2 μg/mL). Instructions are packaged with the container.

### Example 14: Creams for Stimulating the Synthesis of the Extracellular Matrix

In this example, cream pharmaceutical formulations were tested in the combinations and concentrations as shown in Table 2. Creams were used to stimulate factors of extracellular matrix synthesis. AceEBPL-2 is an elastin-like peptide derived from rice.

TABLE 2

| Cream | Ingredient(s) |
|---|---|
| 1 | 50 μg/ml (0.005%) AceEBPL-2, 200 μM Sodium Ascorbate |
| 2 | 50 μg/ml (0.005%) AceEBPL-2, Potassium Iodide 0.025%, |
| | 200 μM Sodium Ascorbate |
| 3 | 50 μg/ml (0.005%) AceEBPL-2, Potassium Iodide 0.025% |
| 4 | 50 μg/ml (0.005%) AceEBPL-2, Tannic Acid 2 μg/ml |
| 5 | 200 μM Sodium L-Ascorbate, 5 μM (0.0002%) Aldosterone |
| 6 | 200 μM Sodium L-Ascorbate, 50 ng/ml IGF-1 |
| 7 | 10 nM Insulin |
| 8 | 10 nM Insulin, 1 mM DMOG (Propyl Hydrolase Inhibitor) |
| 9 | 10 nM Insulin, 2 μg/ml Tannic Acid, 0.025% tincture Iodine |
| 10 | 5 μM (0.0002%) Aldosterone |
| 11 | 5 μM (0.0002%) Aldosterone, 2 μg/ml Copper Sulfate |
| 12 | 5 μM (0.0002%) Aldosterone, 0.0004% Spironolactone |
| 13 | 2 μg/ml Metformin, 0.025% tincture Iodine |
| 14 | 40 nM GLP-1, 0.025% tincture Iodine |
| 15 | 100 ng/ml IGF-1, 0.025% tincture Iodine |
| 16 | 1 ng/ml TGF-beta, 2 μg/ml Tannic Acid |
| 17 | 50 ng/ml Elastin Peptide VGVTAGA (SEQ ID NO: 4) |
| 18 | 50 ng/ml Elastin Peptide VGVAPG (SEQ ID NO: 2) |
| 19 | 5 μg/ml Tannic acid |
| 20 | 2 μg/ml Ellagic Acid, 40 nM GLP-1 |
| 21 | 2 μg/ml Tannic acid |
| 22 | 10 μM (0.0004%) Spironolactone, 5 μM (0.0002%) Aldosterone |

### Example 15: Creams for Protecting Collagen from Degradation

In this example, cream pharmaceutical formulations are tested in the combinations as shown in Table 3. Spironolactone and aldosterone are used to block collagen formation in fibroblasts. Two weeks later, tannic acid is added to the fibroblasts to protect the collagen from further degradation. The same experiment is repeated using a three-week delay period.

TABLE 3

| Cream | Ingredient(s) |
|---|---|
| 1 | EBPL-2, Tannic Acid |
| 2 | EBPL-2, Tannic Acid, 0.25% Potassium Iodide |
| 3 | EBPL-2, Tannic Acid, Dextran |
| 4 | EBPL-2, Tannic Acid, Potassium Iodide, Dextran |
| 5 | EBPL-2, Tannic Acid, Sodium Ascorbate |
| 6 | EBPL-2, Tannic Acid, Sodium Ascorbate, Potassium Iodide |
| 7 | Sodium Ascorbate, Tannic Acid |
| 8 | Tannic Acid, Metformin, Potassium Iodide |

### Embodiments

The following non-limiting embodiments provide illustrative examples of the invention, but do not limit the scope of the invention.

Embodiment 1. A composition comprising: a) a first agent that blocks elastin production; and b) a second agent that promotes elastin production, wherein the second agent is present at a concentration of 0.1 nM to 100 nM.

Embodiment 2. The composition of embodiment 1, wherein the second agent neutralizes blocking of elastin production by the first agent.

Embodiment 3. The composition of embodiment 1, wherein the first agent is a moisturizing agent.

Embodiment 4. The composition of embodiment 3, wherein the moisturizing agent is hyaluronic acid.

Embodiment 5. The composition of embodiment 4, wherein the hyaluronic acid is present in the composition at a concentration of about 10 μg/mL to about 100 μg/mL.

Embodiment 6. The composition of embodiment 3, wherein the moisturizing agent is ferulic acid.

Embodiment 7. The composition of embodiment 6, wherein the ferulic acid is present in the composition at a concentration of about 1 μg/mL to about 15 μg/mL.

Embodiment 8. The composition of embodiment 3, wherein the moisturizing agent is vitamin D.

Embodiment 9. The composition of embodiment 8, wherein the vitamin D is vitamin D₃.

Embodiment 10. The composition of embodiment 8, wherein the vitamin D is present in the composition at a concentration of about 0.01 wt % to about 10 wt %.

Embodiment 11. The composition of embodiment 1, wherein the second agent is a polyphenol.

Embodiment 12. The composition of embodiment 11, wherein the polyphenol is tannic acid.

Embodiment 13. The composition of embodiment 11, wherein the polyphenol is ellagic acid.

Embodiment 14. The composition of embodiment 1, wherein the second agent is present in the composition at a concentration of at most 50 nM.

Embodiment 15. The composition of embodiment 1, wherein the second agent is present in the composition at a concentration of at most 10 nM.

Embodiment 16. The composition of embodiment 1, wherein the second agent is present in the composition at a concentration of at most 2 nM.

Embodiment 17. The composition of embodiment 1, wherein the second agent is present in the composition at a concentration of about 1 μg/mL to about 15 μg/mL.

Embodiment 18. The composition of embodiment 1, further comprising an excipient.

Embodiment 19. The composition of embodiment 1, further comprising a filler.

Embodiment 20. The composition of embodiment 1, further comprising a disintegrant.

Embodiment 21. The composition of embodiment 1, further comprising a preservative agent.

Embodiment 22. The composition of embodiment 1, further comprising an antioxidant.

Embodiment 23. The composition of embodiment 1, further comprising a solvent.

Embodiment 24. The composition of embodiment 1, further comprising a preservative.

Embodiment 25. The composition of embodiment 1, further comprising a suspending agent.

Embodiment 26. The composition of embodiment 1, further comprising a surfactant.

Embodiment 27. The composition of embodiment 1, further comprising a binder.

Embodiment 28. The composition of embodiment 1, further comprising a diluent.

Embodiment 29. The composition of embodiment 1, further comprising a lubricant.

Embodiment 30. The composition of embodiment 1, further comprising a wetting agent.

Embodiment 31. The composition of embodiment 1, further comprising a glidant.

Embodiment 32. The composition of embodiment 1, further comprising a stabilizing agent.

Embodiment 33. The composition of embodiment 1, further comprising a dispersing agent.

Embodiment 34. The composition of embodiment 1, further comprising an elastin-derived peptide.

Embodiment 35. The composition of embodiment 34, wherein the elastin-derived peptide is selected from the group consisting of IGVAPG (SEQ ID NO: 1).

Embodiment 36. The composition of embodiment 34, wherein the elastin-derived peptide is selected from the group consisting of VGVAPG (SEQ ID NO: 2).

Embodiment 37. The composition of embodiment 34, wherein the elastin-derived peptide is selected from the group consisting of VGVTAG (SEQ ID NO: 3).

Embodiment 38. The composition of embodiment 1, further comprising water.

Embodiment 39. The composition of embodiment 1, wherein the composition is substantially free of water.

Embodiment 40. The composition of embodiment 1, wherein the composition is formulated as a cream.

Embodiment 41. The composition of embodiment 1, wherein the composition is formulated as an injectable.

Embodiment 42. A method of enhancing elastin formation comprising: administering to a subject in need thereof a therapeutically-effective amount of a composition, wherein the composition comprises: i) a first agent that blocks elastin production; and ii) a second agent that promotes elastin production, wherein the second agent is present at a concentration of 0.1 nM to 100 nM.

Embodiment 43. The method of embodiment 42, wherein the second agent neutralizes blocking of elastin production by the first agent.

Embodiment 44. The method of embodiment 42, wherein the first agent is a moisturizing agent.

Embodiment 45. The method of embodiment 44, wherein the moisturizing agent comprises hyaluronic acid.

Embodiment 46. The method of embodiment 45, wherein the hyaluronic acid is present in the composition at a concentration of about 10 μg/mL to about 100 μg/mL.

Embodiment 47. The method of embodiment 44, wherein the moisturizing agent comprises ferulic acid.

Embodiment 48. The method of embodiment 47, wherein the ferulic acid is present in the composition at a concentration of about 1 μg/mL to about 15 μg/mL.

Embodiment 49. The method of embodiment 44, wherein the moisturizing agent comprises vitamin D.

Embodiment 50. The method of embodiment 49, wherein the vitamin D is vitamin D₃.

Embodiment 51. The method of embodiment 49, wherein the vitamin D is present in the composition at a concentration of about 0.01 wt % to about 10 wt %.

Embodiment 52. The method of embodiment 42, wherein second agent is a polyphenol.

Embodiment 53. The method of embodiment 52, wherein the polyphenol is tannic acid.

Embodiment 54. The method of embodiment 52, wherein the polyphenol is ellagic acid.

Embodiment 55. The method of embodiment 42, wherein the second agent is present in the composition at a concentration of at most 50 nM.

Embodiment 56. The method of embodiment 42, wherein the second agent is present in the composition at a concentration of at most 10 nM.

Embodiment 57. The method of embodiment 42, wherein the second agent is present in the composition at a concentration of at most 2 nM.

Embodiment 58. The method of embodiment 42, wherein the second agent is present in the composition at a concentration of about 1 μg/mL to about 15 μg/mL.

Embodiment 59. The method of embodiment 42, wherein the composition administered to the subject further comprises an excipient.

Embodiment 60. The method of embodiment 42, wherein the composition administered to the subject further comprises a filler.

Embodiment 61. The method of embodiment 42, wherein the composition administered to the subject further comprises a disintegrant.

Embodiment 62. The method of embodiment 42, wherein the composition administered to the subject further comprises a preservative agent.

Embodiment 63. The method of embodiment 42, wherein the composition administered to the subject further comprises an antioxidant.

Embodiment 64. The method of embodiment 42, wherein the composition administered to the subject further comprises a solvent.

Embodiment 65. The method of embodiment 42, wherein the composition administered to the subject further comprises a preservative.

Embodiment 66. The method of embodiment 42, wherein the composition administered to the subject further comprises a suspending agent.

Embodiment 67. The method of embodiment 42, wherein the composition administered to the subject further comprises a surfactant.

Embodiment 68. The method of embodiment 42, wherein the composition administered to the subject further comprises a binder.

Embodiment 69. The method of embodiment 42, wherein the composition administered to the subject further comprises a diluent.

Embodiment 70. The method of embodiment 42, wherein the composition administered to the subject further comprises a lubricant.

Embodiment 71. The method of embodiment 42, wherein the composition administered to the subject further comprises a wetting agent.

Embodiment 72. The method of embodiment 42, wherein the composition administered to the subject further comprises a glidant.

Embodiment 73. The method of embodiment 42, wherein the composition administered to the subject further comprises a stabilizing agent.

Embodiment 74. The method of embodiment 42, wherein the composition administered to the subject further comprises a dispersing agent.

Embodiment 75. The method of embodiment 42, wherein the composition administered to the subject further comprises an elastin-derived peptide.

Embodiment 76. The method of embodiment 75, wherein the elastin-derived peptide is selected from the group consisting of IGVAPG (SEQ ID NO: 1).

Embodiment 77. The method of embodiment 75, wherein the elastin-derived peptide is selected from the group consisting of VGVAPG (SEQ ID NO: 2).

Embodiment 78. The method of embodiment 75, wherein the elastin-derived peptide is selected from the group consisting of VGVTAG (SEQ ID NO: 3).

Embodiment 79. The method of embodiment 42, wherein the composition administered to the subject further comprises water.

Embodiment 80. The method of embodiment 42, wherein the composition administered to the subject is substantially free of water.

Embodiment 81. The method of embodiment 42, wherein the administering is topical.

Embodiment 82. The method of embodiment 42, wherein the administering is by injection.

Embodiment 83. The method of embodiment 42, wherein the administering enhances elastin formation in the subject by at least 25% as compared to administration of a control, wherein the control is an analogous composition that lacks the second agent.

Embodiment 84. The method of embodiment 42, wherein the administering enhances collagen formation by at least 25% as compared to the control.

Embodiment 85. The method of embodiment 42, wherein the subject is a mammal.

Embodiment 86. The method of embodiment 42, wherein the subject is a human.

Embodiment 87. A method of enhancing elastin formation, the method comprising: administering to a subject in need thereof a therapeutically-effective amount of a composition, wherein the composition comprises: i) a first agent that blocks elastin production; and ii) a second agent that promotes elastin production, wherein the administering enhances elastin formation by at least 25% as compared to administration of a control, wherein the control is an analogous composition that lacks the second agent.

Embodiment 88. A method of enhancing elastin formation, the method comprising: administering to a subject in need thereof a therapeutically-effective amount of a composition, wherein the composition comprises: i) a first agent that blocks elastin production; and ii) a second agent that promotes elastin production, wherein the administering enhances collagen formation by at least 25% as compared to administration of a control, wherein the control is an analogous composition that lacks the second agent.

Embodiment 89. A composition comprising: a) vitamin D; and b) tannic acid, wherein the tannic acid is present at a concentration from 0.1 nM to 100 nM.

1. A topical composition comprising:
a) a first agent that blocks elastin production; and
b) a second agent that promotes elastin production,
wherein the second agent neutralizes blocking of elastin production by the first agent.

2. (canceled)

3. The composition of claim 1, wherein the first agent is a moisturizing agent selected from the group consisting of hyaluronic acid and its pharmaceutically acceptable salts, ferulic acid, and a vitamin D compound.

4. The composition of claim 3, wherein the moisturizing agent is at least two moisturizing agents selected from the group consisting of hyaluronic acid and its pharmaceutically acceptable salts, ferulic acid, and a vitamin D compound.

5-8. (canceled)

9. The composition of claim 3, wherein the vitamin D compound is vitamin D3.

10. (canceled)

11. The composition of claim 1, wherein the second agent is a polyphenol selected from the group consisting of tannic acid and ellagic acid.

12. (canceled)

13. The composition of claim 3, wherein the second agent is a polyphenol selected from the group consisting of ellagic acid and tannic acid.

14-33. (canceled)

34. The composition of claim 1, further comprising an elastin-derived peptide selected from the group consisting of IGVAPG (SEQ ID NO: 1), VGVAPG (SEQ ID NO: 2), and VGVTAG (SEQ ID NO: 3).

35. The composition of claim 3, wherein the elastin-derived peptide is selected from the group consisting of IGVAPG (SEQ ID NO: 1), VGVAPG (SEQ ID NO: 2), and VGVTAG (SEQ ID NO: 3).

36. The composition of claim 11, wherein the elastin-derived peptide is selected from the group consisting of IGVAPG (SEQ ID NO: 1), VGVAPG (SEQ ID NO: 2) and VGVTAG (SEQ ID NO: 3).

37. The composition of claim 13, wherein the elastin-derived peptide is selected from the group consisting of IGVAPG (SEQ ID NO: 1), VGVAPG (SEQ ID NO: 2), VGVTAG (SEQ ID NO: 3).

38-41. (canceled)

42. A method of enhancing elastin formation comprising the step of: administering to a subject in need thereof a therapeutically-effective amount of a topical composition according to claim 1.

43. (canceled)

44. The method of claim 42, wherein the first agent is a moisturizing agent selected from the group consisting of hyaluronic acid and its pharmaceutically acceptable salts, ferulic acid, and a vitamin D compound.

45-49. (canceled)

50. The method of claim 44, wherein the vitamin D is vitamin D3.

51. (canceled)

52. The method of claim 44, wherein second agent is a polyphenol selected from the group consisting of ellagic acid and tannic acid.

53-83. (canceled)

84. The method of claim 44, wherein administering the topical composition according to claim 1 enhances collagen formation by at least 25% as compared to a control.

85-89. (canceled)
